(19) United States
(12) Patent Application Publication
(10) Pub. No.: US 2008/0171318 A1
(43) Pub. Date: Jul. 17, 2008

(54) Epigenetic Methods and Nucleic Acids for the Detection of Lung Cell Proliferative Disorders

(75) Inventors: Achim Plum, Berlin (DE); Tamas Rujan, Berlin (DE); Sabine Maier, Berlin (DE); Ralf Lesche, Berlin (DE); Alexander Graham, Cheshire (GB); Marie South, Cheshire (GB); Iacona Bailey, Wilmington, DE (US)

Correspondence Address: DAVIS WRIGHT TREMAINE, LLP/Seattle, 1201 Third Avenue, Suite 2200, SEATTLE, WA, 98101-3045, US

(73) Assignee: EPIGENOMICS AG, Berlin (DE)

(21) Appl. No.: 11/664,374

(22) PCT Filed: Sep. 30, 2005
(86) PCT No.: PCT/EP05/10611

§ 371 Date: Oct. 16, 2007

(30) Foreign Application Priority Data
Sep. 30, 2004 (EP) 04023300.9

(51) Int. Cl. C12Q 1/68; C07H 21/04

(52) U.S. Cl. 435/6; 536/22.1

(57) ABSTRACT

The invention provides methods, nucleic acids and kits for detecting, classifying and/or distinguishing between or among lung cell proliferative disorders. The invention discloses genomic sequences the methylation patterns of which have utility for the improved detection of and differentiation between said class of disorders, thereby enabling the improved diagnosis and treatment of patients.

### FIELD OF THE INVENTION

The present invention relates to genomic DNA sequences that exhibit altered CpG methylation patterns in lung cell proliferative disease states relative to normal. Particular embodiments provide methods, nucleic acids, nucleic acid arrays and kits useful for detecting, or for detecting and differentiating between or among lung cell proliferative disorders.

### BACKGROUND

DNA methylation. The aetiology of pathogenic states is known to involve modified methylation patterns of individual genes or of the genome. 5-methylcytosine, in the context of CpG dinucleotide sequences, is the most frequent covalently modified base in the DNA of eukaryotic cells, and plays a role in the regulation of transcription, genetic imprinting, and tumourigenesis. The identification and quantification of 5-methylcytosine sites in a specific specimen, or between or among a plurality of specimens, is thus of considerable interest, not only in research, but particularly for the molecular diagnoses of various diseases.

Correlation of aberrant DNA methylation with cancer. Aberrant DNA methylation within CpG ‘islands’ is characterised by hyper- or hypomethylation of CpG dinucleotide sequences leading to abrogation or overexpression of a broad spectrum of genes, and is among the earliest and most common alterations found in, and correlated with human malignancies. Additionally, abnormal methylation has been shown to occur in CpG-rich regulatory elements in intronic and coding parts of genes for certain tumours. In colon cancer, for example, aberrant DNA methylation constitutes one of the most prominent alterations and inactivates many tumour suppresser genes such as p14ARF, p16INK4a, THBS1, MINT2, and MINT31 and DNA mismatch repair genes such as hMLH1.

In contrast to the specific hypermethylation of tumour suppresser genes, an overall hypomethylation of DNA can be observed in tumour cells. This decrease in global methylation can be detected early, far before the development of frank tumour formation. A correlation between hypomethylation and increased gene expression has been determined for many oncogenes.

Lung Cancer incidence and treatment. Lung cancer is the leading cancer-related cause of death with 170,000 people in the USA and one million people worldwide dying every year. About 180,000 new cases are diagnosed every year with 85% of the patients presenting with advanced disease resulting in an overall 5-year survival of only 5% to 15%. In contrast, patients with Stage I disease have a 5-year-survival of about 60%-70%. Cigarette smoking is the major risk factor contributing 80% to the overall attributable risk. Other risks are exposure to asbestos and radon and, to some extend, genetic predisposition.

Lung cancer can be subdivided into four major histological types: adenocarcinoma, squamous cell carcinoma and large cell carcinoma, subsumed under the term non-small cell lung carcinoma (NSCLC), and small cell lung carcinoma (SCLC).

SCLC, which accounts for 20% of the lung cancers, originates from epithelial cells with neuro-endocrine features. It progresses rapidly and usually is detected only in far advanced stages. Chemo- and radiotherapy are the treatments of choice.

NSCLC accounts for the other 80% of lung cancers. While adenocarcinoma arise from alveolar or bronchiolar epithelial or mucin-producing cells, squamous cell carcinoma is derived from bronchial epithelium. Large cell carcinoma is a less differentiated form of adenocarcinoma or squamous cell carcinoma without an apparent glandular of squamous cell phenotype.

Genetic mutations associated with lung cancer. A number of mutations and gene expression changes affecting proto-oncogenes and tumour suppresser genes have been described for lung cancer and some of them could be linked to the early or late stages of tumourigenesis and prognosis for the patient.

Among the most common and earliest genetic alterations detectable in lung cancer is a loss of heterozygosity (LOH) on short arm of chromosome 3 which occurs in 90% of the SCLC tumours and more than 80% of NSCLC lesions. Although several tumour suppresser genes have been mapped to affected regions on 3p such as RARB, FHIT, RASSF1, SEMA3B, and CACNA2D2 it is presently unclear which of these the key players are and in how far they contribute to tumourigenesis.

Activating point mutations in the KRAS2 oncogene are also considered to occur early in NSCLC tumourigenesis and are associated with poor prognosis. Mutations in KRAS2 are found in 15-20% of all NSCLC tumours but practically never occur in SCLC. The mutations mostly occur in codon 12 and strongly correlate with a history of smoking.

Typically, alterations in the signalling pathway regulating the G1/S transition of the cell cycle are altered early in lung cancer development: In 90% of SCLC tumours deletions, nonsense mutations, and splice site mutations are found in RB while expression of CDKN2A coding for p16 is diminished in 70% of NSCLC tumours but rarely in SCLC.

Alterations in the tumour suppresser gene TP53 coding for p53 are the most frequent alterations found in lung cancer but are assumed to occur later in tumour development than 3p alterations, KRAS2 mutations and deregulation of the G1/S transition. Mutations in TP53 are found in about 90% of SCLC and about 50% of NSCLC tumours and certain mutational hotspots correlate with exposure to tobacco smoke.

Overexpression of MYC, either due to gene amplification or transcriptional deregulation, is considered a rather late event in lung tumourigenesis seen in 15-30% of SCLC and 5-10% of NSCLC tumours. It is associated with a poor prognosis for the affected patients.

The anti-apoptotic BCL2 protein is overexpressed in the fast majority of SCLC tumours (75%-95%) but not so common in adenocarcinomas (10%) and squamous cell carcinoma (25%-30%).

The epidermal growth factor receptor gene (EGFR) is commonly overexpressed in NSCLC but not in SCLC and associated with a poor prognosis.

In lung cancer a number of proven or putative tumour suppresser genes were found to be methylated in their promoter region.

Three of the putative tumour suppressor genes that map to chromosome 3p—RARB, RASSF1, FHIT, and SEMA3B were shown to be frequently hypermethylated in lung cancer.

Retinoic acid receptor β (RARB) expression is frequently downregulated in lung cancer and bronchial epithelium of heavy smokers. This downregulation is often mediated by methylation of RARB gene regulatory sequences. In one study, 43% of NSCLC tumours and 62% of SCLC tumours showed hypermethylation at the RARB locus which closely correlated with downregulation of RARB expression.

RASSF1 encodes several transcripts originating from alternative promoters. The expression of one of these transcripts, RASSF1A is frequently lost in lung cancer due to hypermethylation of the respective promoter. RASSF1A methylation is observed in 30-40% of NSCLC tumours and 79-85% in SCLC tumours compared to normal lung tissue.

Aberrant FHIT mRNA and protein expression is also a common phenomenon in lung cancer. In NSCLC these findings correlate well with the hypermethylation of the FHIT promoter (37%).

SemA3B was found to be hypermethylated in 41% of NSCLC tumours. Hypermethylation correlated significantly with LOH at 3p21.3 and most affected tumours lacked of SEMA3B expression.

Another LOH frequently observed in lung cancer is located on 9p21 were CDKN2A coding for p16ink4 maps. Methylation of the remaining allele was frequently observed in NSCLC but rarely in SCLC which is in line with mRNA and protein expression data.

Methylation and lung cancer. Adenomatous polyposis coli (APC) is a tumour suppresser genes well known for its role in familiar and sporadic colon cancer that was also shown to be downregulated by methylation in lung cancer. Depending on the study, 46% to 96% of NSCLC but only 15% of the SCLC tumours show hypermethylation of the APC promoter 1A. Other genes known to be aberrantly methylated in NSCLC include CDH13 (43%-45%), CDH1 (15%-33%), TIMP3 (19%-26%), MGMT (16%-27%), DAPK1 (16%-44%), GSTP1 (7%-12%), CDKN2B/p14 (6%-8%), RUNX3 (3%-24%), IGSF4 (44%), SOCS3 (78%), CHFR (10%), CCND2 (40%), PAX5, LAMB3 (22%-42%), TMS1 (40%), BLU (19%), MYO18B (55%), hMHL (56%), hMSH2, IGFBP3 (62%), SLIT2 (53%), PTEN, COX2/PTGS2 (55%), ESR1, EDNRB, REIC/DKK3, SRBC/CD2, BMP3B (45%), H19/CD59, and HRAS (37%).

Multifactorial approach. Cancer diagnostics has traditionally relied upon the detection of single molecular markers (e.g. gene mutations, elevated PSA levels). Unfortunately, cancer is a disease state in which single markers have typically failed to detect or differentiate many forms of the disease. Thus, assays that recognise only a single marker have been shown to be of limited predictive value, as well be discussed briefly herein. A successful approach currently being pursued in methylation based cancer diagnostics and the screening, diagnosis, and therapeutic monitoring of such diseases is the use of a selection of multiple markers. The multiplexed analytical approach is particularly well suited for cancer diagnostics since cancer is not a simple disease, this multi-factorial “panel” approach is consistent with the heterogeneous nature of cancer, both cytologically and clinically.

Several groups have investigated panels of aberrantly methylated genes in primary lung cancer samples. Zochbauer-Müller et al. (Cancer Res. 2001 Jan. 1; 61(1):249-55) looked at the methylation of 8 genes in 107 NSCLC tumours and matching normal tissue. They found that, at least one of RARB, TIMP3, CDKN2A, CDKN2B, MGMT, DAPK1, CDH1, GSTP1 was aberrantly methylated in 82% of the NSCLC tumours. Significant differences were found for CDKN2A in adenocarcinomas compared to squamous cell carcinoma and in TIMP3, CDKN2A, and DAPK1 in a female vs. male comparison. Harden et al. (Clin Cancer Res. 2003 April; 9(4):1370-5) showed methylation in at least one gene in a panel consisting of CDKN2A, MGMT, GSTP1, APC, and DAPK1 in 81% of Stage I lung cancers. Toyooka et al. (Mol Cancer Ther. 2001 November; 1(1):61-7) compared methylation of CDKN2A, APC, GSTP1, CDH13, MGMT, RARB, CDH1, and RASSF1A in NSCLC, SCLC, and carcinoids and found different patterns of methylation in SCLC and carcinoids compared to NSCLC and between adenocarcinomas and squamous cell carcinomas within the NSCLC group. Yanagawa et al. (Cancer Sci. 2003 July; 94(7):589-92) looked at methylation in a panel consisting of APC, DAPK, CDH1, GSTP1, hMLH1, CDKN2A, RASSF1A, and RUNX3. Of this panel CDKN2A, RASSF1A and RUNX3 were most frequently methylated in tumours but rarely in normal adjacent tissue.

The prognosis for lung cancer patients is strongly correlated with the stage of disease at the time of diagnosis. The five year survival rate for Stage I lung cancer is in the range of 60%-70% while in late stage lung cancer it is reduced to as little as 5%-15%. Thus, early diagnosis is of pivotal importance in the management of this disease. Currently there is no recommendation for screening in lung cancer. Screening trials on long term smokers using chest radiography and sputum cytology failed to reduce lung cancer mortality. Some success has been achieved with low-dose spiral CT scanning. However, this approach is hampered by the high rate of benign nodules detected. Although biomarkers for lung cancer have not yet been clinically validated they could offer interesting alternatives for screening of asymptomatic individuals. Such biomarkers should have a high sensitivity and specificity and appear early enough in the course of the disease to improve prognosis after medical intervention. Most importantly the makers must be detectable in bodily fluids that are easy to obtain to make biomarker analysis feasible in population wide screening programs. Methylation based biomarkers are promising candidates to fulfil these criteria in lung cancer. Methylation changes are early events in lung cancer development and most cancers were shown to have undergone methylation changes in one or several genes. Also, aberrant methylation in lung cancer related genes can be detected in DNA from plasma, serum, sputum, bronchoalveolar lavages, bronchial aspirates, and bronchial brush samples. In one study, Belinsky et al. were able to predict the occurrence of squamous cell carcinoma based on methylation status of CDKN2A/p16 in sputum up to 3 years in advance.

Background of genes associated with the invention. Amphiregulin (AREG) was isolated as a glycoprotein that inhibits growth of certain human tumour cells and stimulates proliferation of human fibroblasts and other normal and tumour cells. The C-terminal half of the protein exhibited striking homology to the epidermal growth factor (EGF) family of proteins. Amphiregulin binds to the EGF receptor with lower affinity than EGF. Overexpression of AREG in NSCLC was reported by several groups.

EGF and ADAM17/TACE are proteases the releases amphiregulin from the cell surface. Serum levels of patients with squamous cell carcinoma have significantly lower levels of IGF1 than patients with adenocarcinoma (Lee D Y, Kim S J, Lee Y C. Serum insulin-like growth factor (IGF)-I and IGF-binding proteins in lung cancer patients. J Korean Med Sci. 1999 August; 14(4):401-4). IGF1 has been reported as aberrantly methylated in cancers, but not in lung cancer Schneid H, Seurin D, Noguiez P, Le Bouc Y. Abnormalities of insulin-like growth factor (IGF-I and IGF-II) genes in human tumor tissue. Growth Regul. 1992 March; 2(1):45-54.

Neuregulin encoded by NRG is another growth factor of the EGF family. Neuregulin is highly expressed in many regions of the brain. Using a tagged version of the NRG3 EGF-like domain, Zhang et al. (1997) demonstrated that NRG3 can bind to the extracellular domain of the ERBB4 receptor tyrosine kinase. Later, Hijazi et al. reported that NRG3 is also able to activate other receptors of the erbb family including EGFR. They further found that recombinant NRG3 altered the growth of human breast cancer cells and concluded that NRG3 is potentially a regulator of normal and malignant breast epithelial cells.

RASGRP1 and RASGRP2 (RAS guanyl nucleotide-releasing protein 1/2) both belong to a family of genes characterised by the presence of a Ras superfamily guanine nucleotide exchange factor (GEF) domain. As indicated by their name, they are capable of activating Ras proteins by exchanging GDP with GTP.

Recently, Bivona et al. demonstrated that in response to Src-dependent activation of phospholipase C gamma1, RASGRP1 translocates to the Golgi where it activates RAS proteins. They concluded that activation of Ras on Golgi has important biological consequences and proceeds through a pathway distinct from the one that activates Ras on the plasma membrane. RASGRP1 can counteract signalling of activated KRAS or HRAS by specific activation of RAP1A. KRAS2 is frequently mutated in NSCLC. Hypermethylation of RASGRP1 or RASGRP2 was not reported to date.

RASGRF1 encodes the ras protein-specific guanine nucleotide releasing-factor 1, a 55 kDA protein that triggers the release of bound GDP specifically from RAS but not from RAP1A. Release of GDP is a prerequisite for RAS to return into its active state with GTP bound. In mice RASGRF1 was shown to be imprinted and expression was dependent on methylation of a differential methylation domain about 30 kb upstream of the gene.

FOXF1 encodes the transcription factor forkhead box f1 also known as forkhead-related activator 1. There are no previous reports on methylation of this gene in lung cancer. Mice with heterozygous deletion of the Foxf1 gene show severely impaired lung development (Mahlapuu M, Enerback S, Carlsson P. Haploinsufficiency of the forkhead gene Foxf1, a target for sonic hedgehog signalling, causes lung and foregut malformations. Development. 2001 June; 128(12):2397-406). FOXF1 activates the transcription of lung-specific genes such as SPB, the gene encoding surfactant protein b and CC10 encoding the 10 kDa clara cell protein.

MDR1/ABCB1. The ABCB1 gene is better known as MDR1 (multidrug resistance gene 1). It encodes a large transmembrane protein that is an integral part of the blood-brain barrier and functions as a pump transporting a variety of drugs from the brain back into the blood. Drug resistance in acute myeloid leukaemia is characterised by the expression of the MDR1 gene product. Kantharidis et al. (1997) found a tight correlation between the MDR phenotype and demethylation of the 5′ region of the MDR1 gene in a human T cell leukaemia cell line suggesting that the MDR phenotype may be acquired as a result of changes in methylation of the MDR1 promoter.

AKT2 is a putative cellular homologue of the v-akt oncogene encoding an isoform of the phosphoinositide-dependent serine-threonine protein kinase Akt. However, Cheng et al. showed that AKT2 was amplified and overexpressed in 2 of 8 ovarian carcinoma cell lines and 2 of 15 primary ovarian tumours. Later, the same group demonstrated that AKT2 is also amplified in approximately 10% of pancreatic carcinomas. Furthermore, tumourigenicity in nude mice was markedly reduced in the pancreatic cancer cells expressing antisense AKT2 RNA (Cheng, J. Q.; Ruggeri, B.; Klein, W. M.; Sonoda, G.; Altomare, D. A.; Watson, D. K.; Testa, J. R.: Amplification of AKT2 in human pancreatic cancer cells and inhibition of AKT2 expression and tumourigenicity by antisense RNA. Proc. Nat. Acad. Sci. 93: 3636-3641, 1996). These data suggested that overexpression of AKT2 contributes to the malignant phenotype of a subset of human ductal pancreatic cancers. DNA methylation in NSCLC or other cancers is not described for this gene so far.

PIK3CA encodes the catalytic subunit of phosphatidylinositol 3-kinase. The kinase is composed of two subunits with 85 kDa and 110 kDa. The 85 kD subunit lacks PI3-kinase activity and acts as an adapter, coupling the 110-kDa catalytic subunit (p110) to activated protein tyrosine kinases via IRS1/2. PI3-kinase can also be activated via RAS. P110 can activate AKT kinase via phosphorylation of phosphatidylinositol(4,5)phosphate in the cell membrane or other pathways by direct interaction with cytosolic proteins. Activation of PI3K in neoplastic cells was shown to inhibit apoptosis and enhance cell survival, cell cycle, and transformation. Gain-of-function mutations were found in some colon, brain and gastrointestinal tumours but rarely in lung and breast cancer. Amplification was reported as an other mechanism of oncogene activation in some cancers. The PIK3CA promoter is not associated with a CpG-island and there are no reports on differential methylation of PIK3CA in lung or other cancers. PAK7 belongs to the PAK B subfamily of p21-activated kinases and is expressed at high levels in brain and lower levels in other tissues.

The gene BCL2, is known to be over expressed in some NSCLCs, LOH and concomitant hypermethylation of the gene locus has also been reported (Cancer Res. 1996 Apr. 15; 56(8):1886-91). ESR1 encodes the estrogen receptor α, a nuclear hormone receptor that upon binding of estrogen translocates from the cytoplasm to the cell nucleus and can directly act as a transcription factor. Although epidemiology suggests a possible role for estrogens in lung cancer, there are conflicting results on the expression of the receptor protein in NSCLC. Nevertheless, cell line experiments suggest functionality of estrogen signalling in NSCLC cells. ESR1 promoter hypermethylation was previously found in a subset of lung tumours and was inversely correlated to the exposure to tobacco smoke. The hypermethylation of the ESR1 promoter in some NSCLC tumours could be confirmed in this study. In addition to the typical activating point mutations in the proto-oncogene HRAS in many cancers, methylation is also described for the HRAS gene. Vachtenheim et al. found hypomethylation of CCGG sites in the 3′ region of the gene and that was associated with the loss of the second HRAS allele in non-small cell lung cancer. GSTP1 encodes the glutathione s-transferase pi a detoxifying enzyme typically overexpressed in multidrug-resistant cells. Numerous authors report hypermethylation of the GSTP1 promoter in 1%-12% of the NSCLC tumours. IGF1 encoding the insulin-like growth factor was reported in to be aberrantly methylated in some primary tumours, but not in NSCLC. The same applies for FOS which was shown to be aberrantly methylated in gastrointestinal cancer, hepatocellular cancer, and gliomas but not analysed in lung cancer. For STAT1 there is indirect evidence for a regulation by methylation that is based on the finding that STAT1 expression can be induced by inhibition of DNA methylation in colon cancer cell lines but no previous reports on methylation of STAT1 in lung cancer were found. Similarly, SHC1 was reported to be regulated by two alternative promoters, one of which could be activated by DNA methylation inhibitors.

Development of medical tests. Key to the successful implementation of a panel approach to methylation based diagnostic tests is the design and development of optimised panels of markers that can characterise and distinguish disease states. This patent application describes an efficient and unique panel of genes the methylation analysis of one or a combination of the members of the panel enabling the detection of lung cell proliferative disorders with a particularly high sensitivity, specificity and/or predictive value.

Two key evaluative measures of any medical screening or diagnostic test are its sensitivity and specificity, which measure how well the test performs to accurately detect all affected individuals without exception, and without falsely including individuals who do not have the target disease (predictive value). Historically, many diagnostic tests have been criticised due to poor sensitivity and specificity.

A true positive (TP) result is where the test is positive and the condition is present. A false positive (FP) result is where the test is positive but the condition is not present. A true negative (TN) result is where the test is negative and the condition is not present. A false negative (FN) result is where the test is negative but the condition is not present.

Sensitivity=TP/(TP+FN)

Specificity=TN/(FP+TN)

Predictive value=TP/(TP+FP)

Sensitivity is a measure of a test's ability to correctly detect the target disease in an individual being tested. A test having poor sensitivity produces a high rate of false negatives, i.e., individuals who have the disease but are falsely identified as being free of that particular disease. The potential danger of a false negative is that the diseased individual will remain undiagnosed and untreated for some period of time, during which the disease may progress to a later stage wherein treatments, if any, may be less effective. An example of a test that has low sensitivity is a protein-based blood test for HIV. This type of test exhibits poor sensitivity because it fails to detect the presence of the virus until the disease is well established and the virus has invaded the bloodstream in substantial numbers. In contrast, an example of a test that has high sensitivity is viral-load detection using the polymerase chain reaction (PCR). High sensitivity is achieved because this type of test can detect very small quantities of the virus. High sensitivity is particularly important when the consequences of missing a diagnosis are high.

Specificity, on the other hand, is a measure of a test's ability to identify accurately patients who are free of the disease state. A test having poor specificity produces a high rate of false positives, i.e., individuals who are falsely identified as having the disease. A drawback of false positives is that they force patients to undergo unnecessary medical procedures treatments with their attendant risks, emotional and financial stresses, and which could have adverse effects on the patient's health. A feature of diseases which makes it difficult to develop diagnostic tests with high specificity is that disease mechanisms, particularly in cancer, often involve a plurality of genes and proteins. Additionally, certain proteins may be elevated for reasons unrelated to a disease state. An example of a test that has high specificity is a gene-based test that can detect a p53 mutation. Specificity is important when the cost or risk associated with further diagnostic procedures or further medical intervention are very high.

### SUMMARY OF THE INVENTION

The present invention provides novel methods for detecting and/or differentiating between lung cell proliferative disorders, in particular lung cancer.

The invention solves a longstanding need in the art for improved means of cancer diagnostics and classification by providing a panel of genes and genomic sequences thereof (according to the sequence listing), the methylation status of CpG positions of these genes and/or their promoter regions being indicative of lung cell proliferative disorders (in particular cancer) or features thereof. Preferred selections and combinations of genes are provided, the methylation analysis of which enable the differentiation and detection of various classes of lung cell proliferative disorders, namely:
Detection of lung cell proliferative disorders, preferably non small cell lung cancer (hereinafter also referred to as NSCLC).
Molecular classification (hereinafter also referred to as differentiation) of lung squamous cell carcinoma and lung adenocarcinoma.
Detection of lung squamous cell carcinoma.
Detection of lung adenocarcinoma.

In order to enable this analysis, the invention provides a method for the analysis of biological samples for genomic methylation associated with the development of lung cell proliferative disorders. Said method is characterised in that at least one nucleic acid, or a fragment thereof, from the group consisting of SEQ ID NO: 1 to SEQ ID NO: 56 is/are contacted with a reagent or series of reagents capable of distinguishing between methylated and non methylated CpG dinucleotides within the genomic sequence, or sequences of interest.

The present invention provides a method for ascertaining genetic and/or epigenetic parameters of genomic DNA. The method has utility for the improved diagnosis, differentiation and treatment of lung cell proliferative disorders, more specifically by enabling the improved identification of and differentiation between subclasses of said disorder. The invention presents several improvements over the state of the art. Although aberrant methylation as a hallmark of lung cancer is known there are currently no methylation markers that are suitably accurate and robust for use in a clinically approved (e.g. U.S. FDA) publicly available assay.

The test sample may be from any suitable source, such as cell lines, histological slides, biopsies, paraffin-embedded tissue, bodily fluids, sputum, blood plasma, blood serum, whole blood, isolated blood cells, cells isolated from the blood and all possible combinations thereof.

Specifically, the present invention provides a method for detecting lung cell proliferative disorders, comprising: obtaining a biological sample comprising genomic nucleic acid(s); contacting the nucleic acid(s), or a fragment thereof, with one reagent or a plurality of reagents sufficient for distinguishing between methylated and non methylated CpG dinucleotide sequences within a target sequence of the subject nucleic acid, wherein the target sequence comprises, or hybridises under stringent conditions to, a sequence comprising at least 16 contiguous nucleotides of SEQ ID NO: 1 to SEQ ID NO: 56, said contiguous nucleotides comprising at least one CpG dinucleotide sequence; and determining, based at least in part on said distinguishing, the methylation state of at least one target CpG dinucleotide sequence, or an average, or a value reflecting an average methylation state of a plurality of target CpG dinucleotide sequences. Preferably, distinguishing between methylated and non methylated CpG dinucleotide sequences within the target sequence comprises methylation state-dependent conversion or non-conversion of at least one such CpG dinucleotide sequence to the corresponding converted or non-converted dinucleotide sequence within a sequence selected from the group consisting of SEQ ID NO: 236 to SEQ ID NO: 347, and contiguous regions thereof corresponding to the target sequence.

Additional embodiments provide a method for the detection of lung cell proliferative disorders, comprising: obtaining a biological sample having subject genomic DNA; extracting the genomic DNA; treating the genomic DNA, or a fragment thereof, with one or more reagents to convert 5-position unmethylated cytosine bases to uracil or to another base that is detectably dissimilar to cytosine in terms of hybridisation properties; contacting the treated genomic DNA, or the treated fragment thereof, with an amplification enzyme and at least two primers comprising, in each case a contiguous sequence at least 9 nucleotides in length that is complementary to, or hybridises under moderately stringent or stringent conditions to a sequence selected from the group consisting SEQ ID NO: 236 to SEQ ID NO: 347, and complements thereof, wherein the treated DNA or the fragment thereof is either amplified to produce an amplificate, or is not amplified; and determining, based on a presence or absence of, or on a property of said amplificate, the methylation state of at least one CpG dinucleotide sequence selected from the group consisting of SEQ ID NO: 1 to SEQ ID NO: 56, or an average, or a value reflecting an average methylation state of a plurality of CpG dinucleotide sequences thereof. Preferably, at least one such hybridising nucleic acid molecule or peptide nucleic acid molecule is bound to a solid phase. Preferably, determining comprises use of at least one method selected from the group consisting of: hybridising at least one nucleic acid molecule comprising a contiguous sequence at least 9 nucleotides in length that is complementary to, or hybridises under moderately stringent or stringent conditions to a sequence selected from the group consisting of SEQ ID NO: 236 to SEQ ID NO: 347, and complements thereof; hybridising at least one nucleic acid molecule, bound to a solid phase, comprising a contiguous sequence at least 9 nucleotides in length that is complementary to, or hybridises under moderately stringent or stringent conditions to a sequence selected from the group consisting of SEQ ID NO: 236 to SEQ ID NO: 347, and complements thereof; hybridising at least one nucleic acid molecule comprising a contiguous sequence at least 9 nucleotides in length that is complementary to, or hybridises under moderately stringent or stringent conditions to a sequence selected from the group consisting of SEQ ID NO: 236 to SEQ ID NO: 347, and complements thereof, and extending at least one such hybridised nucleic acid molecule by at least one nucleotide base; and sequencing of the amplificate.

Further embodiments provide a method for the analysis of lung cell proliferative disorders, comprising: obtaining a biological sample having subject genomic DNA; extracting the genomic DNA; contacting the genomic DNA, or a fragment thereof, comprising one or more sequences selected from the group consisting of SEQ ID NO: 1 to SEQ ID NO: 56 or a sequence that hybridises under stringent conditions thereto, with one or more methylation-sensitive restriction enzymes, wherein the genomic DNA is either digested thereby to produce digestion fragments, or is not digested thereby; and determining, based on a presence or absence of, or on property of at least one such fragment, the methylation state of at least one CpG dinucleotide sequence of one or more sequences selected from the group consisting of SEQ ID NO: 1 to SEQ ID NO: 56, or an average, or a value reflecting an average methylation state of a plurality of CpG dinucleotide sequences thereof. Preferably, the digested or undigested genomic DNA is amplified prior to said determining.

Additional embodiments provide novel genomic and chemically modified nucleic acid sequences, as well as oligonucleotides and/or PNA-oligomers for analysis of cytosine methylation patterns within sequences from the group consisting of SEQ ID NO: 1 to SEQ ID NO: 56.

### BRIEF DESCRIPTION OF THE DRAWINGS

FIG. 1 shows a ranked matrix of data obtained according to the microarray analysis of Example 1 of CpG methylation differences between the two classes of tissues, (lung cancer and NAT) using univariate analysis. The most significant CpG positions are at the bottom of the matrix with significance decreasing towards the top. Red indicates total methylation at a given CpG position, green represents no methylation at the particular position. Each row represents one specific CpG position within a gene and each column represents the methylation profile for the different CpGs for one sample. The scale at the bottom of the figure enables the relative calibration of methylation levels at each position of the matrix from −2 (total non methylation) to 2 (complete methylation). SEQ ID NOs: of the relevant detection oligonucleotides are shown on the left hand side of the matrix.

FIG. 2 shows a ranked matrix of data obtained according to the microarray analysis of Example 1 of CpG methylation differences between the two classes of tissues, (lung cancer and NAT) using multivariate analysis. The most significant CpG positions are at the bottom of the matrix with significance decreasing towards the top. Red indicates total methylation at a given CpG position, green represents no methylation at the particular position. Each horizontal block represents one region of interest of a gene and each row thereof a specific CpG position within said region of interest. Each column represents the methylation profile for the different CpGs for one sample. The scale at the bottom of the figure enables the relative calibration of methylation levels at each position of the matrix from −2 (total non methylation) to 2 (complete methylation).

FIG. 3 shows a ranked matrix of data obtained according to the microarray analysis of Example 1 of CpG methylation differences between the two classes of tissues, (lung adenocarcinoma and lung squamous cell carcinoma) using univariate analysis. The most significant CpG positions are at the bottom of the matrix with significance decreasing towards the top. Red indicates total methylation at a given CpG position, green represents no methylation at the particular position. Each row represents one specific CpG position within a gene and each column represents the methylation profile for the different CpGs for one sample. The scale at the bottom of the figure enables the relative calibration of methylation levels at each position of the matrix from −2 (total non methylation) to 2 (complete methylation). SEQ ID NOs: of the relevant detection oligonucleotides are shown on the left hand side of the matrix.

FIG. 4 shows a ranked matrix of data obtained according to the microarray analysis of Example 1 of CpG methylation differences between the two classes of tissues, (lung adenocarcinoma and lung squamous cell carcinoma) using multivariate analysis. The most significant CpG positions are at the bottom of the matrix with significance decreasing towards the top. Red indicates total methylation at a given CpG position, green represents no methylation at the particular position. Each horizontal block represents one region of interest of a gene and each row thereof a specific CpG position within said region of interest. Each column represents the methylation profile for the different CpGs for one sample. The scale at the bottom of the figure enables the relative calibration of methylation levels at each position of the matrix from −2 (total non methylation) to 2 (complete methylation).

FIG. 5 shows a ranked matrix of data obtained according to the microarray analysis of Example 1 of CpG methylation differences between the two classes of tissues, (lung adenocarcinoma and NAT) using univariate analysis. The most significant CpG positions are at the bottom of the matrix with significance decreasing towards the top. Red indicates total methylation at a given CpG position, green represents no methylation at the particular position. Each row represents one specific CpG position within a gene and each column represents the methylation profile for the different CpGs for one sample. The scale at the bottom of the figure enables the relative calibration of methylation levels at each position of the matrix from −2 (total non methylation) to 2 (complete methylation). SEQ ID NOs: of the relevant detection oligonucleotides are shown on the left hand side of the matrix.

FIG. 6 shows a ranked matrix of data obtained according to the microarray analysis of Example 1 of CpG methylation differences between the two classes of tissues, (lung adenocarcinoma and NAT) using multivariate analysis. The most significant CpG positions are at the bottom of the matrix with significance decreasing towards the top. Red indicates total methylation at a given CpG position, green represents no methylation at the particular position. Each horizontal block represents one region of interest of a gene and each row thereof a specific CpG position within said region of interest. Each column represents the methylation profile for the different CpGs for one sample. The scale at the bottom of the figure enables the relative calibration of methylation levels at each position of the matrix from −2 (total non methylation) to 2 (complete methylation).

FIG. 7 shows a ranked matrix of data obtained according to the microarray analysis of Example 1 of CpG methylation differences between the two classes of tissues, (lung squamous cell carcinoma and NAT) using univariate analysis. The most significant CpG positions are at the bottom of the matrix with significance decreasing towards the top. Red indicates total methylation at a given CpG position, green represents no methylation at the particular position. Each row represents one specific CpG position within a gene and each column represents the methylation profile for the different CpGs for one sample. SEQ ID NOs: of the relevant detection oligonucleotides are shown on the left hand side of the matrix.

FIG. 8 shows a ranked matrix of data obtained according to the microarray analysis of Example 1 of CpG methylation differences between the two classes of tissues, (lung squamous cell carcinoma and NAT) using multivariate analysis. The most significant CpG positions are at the bottom of the matrix with significance decreasing towards the top. Red indicates total methylation at a given CpG position, green represents no methylation at the particular position. Each horizontal block represents one region of interest of a gene and each row thereof a specific CpG position within said region of interest. Each column represents the methylation profile for the different CpGs for one sample. The scale at the bottom of the figure enables the relative calibration of methylatin levels at each position of the matrix from −2 (total non methylation) to 2 (complete methylation).

FIGS. 9 to 16 provide greyscale versions of FIGS. 1 to 8 respectively.

### DETAILED DESCRIPTION OF THE INVENTION

### Definitions

The term “CpG island” refers to a contiguous region of genomic DNA that satisfies the criteria of (1) having a frequency of CpG dinucleotides corresponding to an “Observed/Expected Ratio”>0.6, and (2) having a “GC Content”>0.5.CpG islands are typically, but not always, between about 0.2 to about 1 kb, or to about 2 kb in length.

The term “methylation state” or “methylation status” refers to the presence or absence of 5-methylcytosine (“5-mCyt”) at one or a plurality of CpG dinucleotides within a DNA sequence. Methylation states at one or more particular CpG methylation sites (each having two CpG CpG dinucleotide sequences) within a DNA sequence include “unmethylated,” “fully-methylated” and “hemi-methylated.”

The term “hemi-methylation” or “hemimethylation” refers to the methylation state of a double stranded nucleic acid, where only one strand thereof is methylated.

The term ‘AUC’ as used herein is an abbreviation of “area under the curve”. In particular it refers to the area under a Receiver Operating Characteristic (ROC) curve. The ROC curve is a plot of the true positive rate against the false positive rate for the different possible cutpoints of a diagnostic test. It shows the trade-off between sensitivity and specificity depending on the selected cut-off point (any increase in sensitivity will be accompanied by a decrease in specificity). The area under an ROC curve (AUC) is a measure of the accuracy of a diagnostic test (the larger the area the better, optimum is 1, a random test would have a ROC curve lying on the diagonal with an area of 0.5; for reference: J. P. Egan. Signal Detection Theory and ROC Analysis, Academic Press, New York, 1975).

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample. Alternatively the term “hypermethylation” may be defined as relative to a cut-off point. It is particularly preferred that said cut-off point is between 3% and 5%.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample. Alternatively the term “hypomethylation” may be defined as relative to a cut-off point. It is particularly preferred that said cut-off point is between 3% and 5%.

The term “microarray” refers broadly to both “DNA microarrays,” and “DNA chip(s)”, as recognised in the art, encompasses all art-recognised solid supports, and encompasses all methods for affixing nucleic acid molecules thereto or synthesis of nucleic acids thereon.

“Genetic parameters” are mutations and polymorphisms of genes and sequences further required for their regulation. To be designated as mutations are, in particular, insertions, deletions, point mutations, inversions and polymorphisms and, particularly preferred, SNPs (single nucleotide polymorphisms).

“Epigenetic parameters” are, in particular, cytosine methylations. Further epigenetic parameters include, for example, the acetylation of histones which, however, cannot be directly analysed using the described method but which, in turn, correlate with the DNA methylation.

The term “bisulfite reagent” refers to a reagent comprising bisulfite, disulfite, hydrogen sulfite or combinations thereof, useful as disclosed herein to distinguish between methylated and unmethylated CpG dinucleotide sequences.

The term “Methylation assay” refers to any assay for determining the methylation state of one or more CpG dinucleotide sequences within a sequence of DNA.

The terms “MS.AP-PCR” or “AP-PCR” (Methylation-Sensitive Arbitrarily-Primed Polymerase Chain Reaction) refer to the art-recognised technology that allows for a global scan of the genome using CG-rich primers to focus on the regions most likely to contain CpG dinucleotides, and described by Gonzalgo et al., Cancer Research 57:594-599, 1997.

The term “MethyLight™” refers to the art-recognised fluorescence-based real-time PCR technique described by Eads et al., Cancer Res. 59:2302-2306, 1999.

The term “HeavyMethyl™” assay, in the embodiment thereof implemented herein, refers to an assay, wherein methylation specific blocking probes (also referred to herein as blockers) covering CpG positions between, or covered by the amplification primers enable methylation-specific selective amplification of a nucleic acid sample.

The term “HeavyMethyl™ MethyLight™” assay, in the embodiment thereof implemented herein, refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognised assay described by Gonzalgo and Jones, Nucleic Acids Res. 25:2529-2531, 1997.

The term “MSP” (Methylation-specific PCR) refers to the art-recognised methylation assay described by Herman et al. Proc. Natl. Acad. Sci. USA 93:9821-9826, 1996, and by U.S. Pat. No. 5,786,146.

The term “COBRA” (Combined Bisulfite Restriction Analysis) refers to the art-recognised methylation assay described by Xiong and Laird, Nucleic Acids Res. 25:2532-2534, 1997.

The term “MCA” (Methylated CpG Island Amplification) refers to the methylation assay described by Toyota et al., Cancer Res. 59:2307-12, 1999, and in WO 00/26401A1.

The term “hybridisation” is to be understood as a bond of an oligonucleotide to a complementary sequence along the lines of the Watson-Crick base pairings in the sample DNA, forming a duplex structure.

“Stringent hybridisation conditions,” as defined herein, involve hybridising at 68° C. in 5×SSC/5×Denhardt's solution/1.0% SDS, and washing in 0.2×SSC/0.1% SDS at room temperature, or involve the art-recognised equivalent thereof (e.g., conditions in which a hybridisation is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognised equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley and Sons, N.Y.) at Unit 2.10.

The terms “array SEQ ID NO,” “composite array SEQ ID NO,” or “composite array sequence” refer to a sequence, hypothetical or otherwise, consisting of a head-to-tail (5′ to 3′) linear composite of all individual contiguous sequences of a subject array (e.g., a head-to-tail composite of SEQ ID NO:1-71, in that order).

The terms “array SEQ ID NO node,” “composite array SEQ ID NO node,” or “composite array sequence node” refer to a junction between any two individual contiguous sequences of the “array SEQ ID NO,” the “composite array SEQ ID NO,” or the “composite array sequence.”

In reference to composite array sequences, the phrase “contiguous nucleotides” refers to a contiguous sequence region of any individual contiguous sequence of the composite array, but does not include a region of the composite array sequence that includes a “node,” as defined herein above.

The present invention provides for molecular genetic markers that have novel utility for the analysis of methylation patterns associated with the development of lung cell proliferative disorders. Said markers may be used for detecting or distinguishing between lung cell proliferative disorders, thereby providing improved means for the detection, classification and therapy of said disorders.

Bisulfite modification of DNA is an art-recognised tool used to assess CpG methylation status. 5-methylcytosine is the most frequent covalent base modification in the DNA of eukaryotic cells. It plays a role, for example, in the regulation of the transcription, in genetic imprinting, and in tumourigenesis. Therefore, the identification of 5-methylcytosine as a component of genetic information is of considerable interest. However, 5-methylcytosine positions cannot be identified by sequencing, because 5-methylcytosine has the same base pairing behaviour as cytosine. Moreover, the epigenetic information carried by 5-methylcytosine is completely lost during, e.g., PCR amplification.

The most frequently used method for analysing DNA for the presence of 5-methylcytosine is based upon the specific reaction of bisulfite with cytosine whereby, upon subsequent alkaline hydrolysis, cytosine is converted to uracil which corresponds to thymine in its base pairing behaviour. Significantly, however, 5-methylcytosine remains unmodified under these conditions. Consequently, the original DNA is converted in such a manner that methylcytosine, which originally could not be distinguished from cytosine by its hybridisation behaviour, can now be detected as the only remaining cytosine using standard, art-recognised molecular biological techniques, for example, by amplification and hybridisation, or by sequencing. All of these techniques are based on differential base pairing properties, which can now be fully exploited.

The prior art, in terms of sensitivity, is defined by a method comprising enclosing the DNA to be analysed in an agarose matrix, thereby preventing the diffusion and renaturation of the DNA (bisulfite only reacts with single-stranded DNA), and replacing all precipitation and purification steps with fast dialysis (Olek A, et al., A modified and improved method for bisulfite based cytosine methylation analysis, Nucleic Acids Res. 24:5064-6, 1996). It is thus possible to analyse individual cells for methylation status, illustrating the utility and sensitivity of the method. An overview of art-recognised methods for detecting 5-methylcytosine is provided by Rein, T., et al., Nucleic Acids Res., 26:2255, 1998.

The bisulfite technique, barring few exceptions (e.g., Zeschnigk M, et al., Eur J Hum Genet. 5:94-98, 1997), is currently only used in research. In all instances, short, specific fragments of a known gene are amplified subsequent to a bisulfite treatment, and either completely sequenced (Olek and Walter, Nat. Genet. 1997 17:275-6, 1997), subjected to one or more primer extension reactions (Gonzalgo and Jones, Nucleic Acids Res., 25:2529-31, 1997; WO 95/00669; U.S. Pat. No. 6,251,594) to analyse individual cytosine positions, or treated by enzymatic digestion (Xiong and Laird, Nucleic Acids Res., 25:2532-4, 1997). Detection by hybridization has also been described in the art (Olek et al., WO 99/28498). Additionally, use of the bisulfite technique for methylation detection with respect to individual genes has been described (Grigg and Clark, Bioassays, 16:431-6, 1994; Zeschnigk M, et al., Hum Mol Genet., 6:387-95, 1997; Feil R, et al., Nucleic Acids Res., 22:695-, 1994; Martin V, et al., Gene, 157:261-4, 1995; WO 9746705 and WO 9515373).

The present invention provides for the use of the bisulfite technique, in combination with one or more methylation assays, for determination of the methylation status of CpG dinucleotide sequences within sequences from the group consisting of SEQ ID NO: 1 to SEQ ID NO: 56. According to the present invention, determination of the methylation status of CpG dinucleotide sequences within sequences from the group consisting of SEQ ID NO: 1 to SEQ ID NO: 56 has diagnostic and prognostic utility.

Methylation Assay Procedures. Various methylation assay procedures are known in the art, and can be used in conjunction with the present invention. These assays allow for determination of the methylation state of one or a plurality of CpG dinucleotides (e.g., CpG islands) within a DNA sequence. Such assays involve, among other techniques, DNA sequencing of bisulfite-treated DNA, PCR (for sequence-specific amplification), Southern blot analysis, and use of methylation-sensitive restriction enzymes.

For example, genomic sequencing has been simplified for analysis of DNA methylation patterns and 5-methylcytosine distribution by using bisulfite treatment (Frommer et al., Proc. Natl. Acad. Sci. USA 89:1827-1831, 1992). Additionally, restriction enzyme digestion of PCR products amplified from bisulfite-converted DNA is used, e.g., the method described by Sadri and Hornsby (Nucl. Acids Res. 24:5058-5059, 1996), or COBRA (Combined Bisulfite Restriction Analysis) (Xiong and Laird, Nucleic Acids Res. 25:2532-2534, 1997).

COBRA analysis is a quantitative methylation assay useful for determining DNA methylation levels at specific gene loci in small amounts of genomic DNA (Xiong and Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the CpG islands of interest, followed by restriction endonuclease digestion, gel electrophoresis, and detection using specific, labelled hybridisation probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples. Typical reagents (e.g., as might be found in a typical COBRA-based kit) for COBRA analysis may include, but are not limited to: PCR primers for specific gene (or bisulfite treated DNA sequence or CpG island); restriction enzyme and appropriate buffer; gene-hybridisation oligo; control hybridisation oligo; kinase labelling kit for oligo probe; and labelled nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

Preferably, assays such as “MethyLight™” (a fluorescence-based real-time PCR technique) (Eads et al., Cancer Res. 59:2302-2306, 1999), Ms-SNuPE (Methylation-sensitive Single Nucleotide Primer Extension) reactions (Gonzalgo and Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with other of these methods.

The MethyLight™ assay is a high-throughput quantitative methylation assay that utilises fluorescence-based real-time PCR (TaqMan™) technology that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed either in an “unbiased” (with primers that do not overlap known CpG methylation sites) PCR reaction, or in a “biased” (with PCR primers that overlap known CpG dinucleotides) reaction. Sequence discrimination can occur either at the level of the amplification process or at the level of the fluorescence detection process, or both.

The MethyLight™ assay may be used as a quantitative test for methylation patterns in the genomic DNA sample, wherein sequence discrimination occurs at the level of probe hybridisation. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique), or with oligonucleotides covering potential methylation sites.

The MethyLight™ process can by used with a “TaqMan®” probe in the amplification process. For example, double-stranded genomic DNA is treated with sodium bisulfite and subjected to one of two sets of PCR reactions using TaqMan® probes; e.g., with either biased primers and TaqMan® probe, or unbiased primers and TaqMan® probe. The TaqMan® probe is dual-labelled with fluorescent “reporter” and “quencher” molecules, and is designed to be specific for a relatively high GC content region so that it melts out at about 10° C. higher temperature in the PCR cycle than the forward or reverse primers. This allows the TaqMan® probe to remain fully hybridised during the PCR annealing/extension step. As the Taq polymerase enzymatically synthesises a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific gene (or bisulfite treated DNA sequence or CpG island); TaqMan® probes; optimised PCR buffers and deoxynucleotides; and Taq polymerase.

The Ms-SNuPE technique is a quantitative method for assessing methylation differences at specific CpG sites based on bisulfite treatment of DNA, followed by single-nucleotide primer extension (Gonzalgo and Jones, Nucleic Acids Res. 25:2529-2531, 1997). Briefly, genomic DNA is reacted with sodium bisulfite to convert umethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. Small amounts of DNA can be analysed (e.g., microdissected pathology sections), and it avoids utilisation of restriction enzymes for determining the methylation status at CpG sites.

Typical reagents (e.g., as might be found in a typical Ms-SNuPE-based kit) for Ms-SNuPE analysis may include, but are not limited to: PCR primers for specific gene (or bisulfite treated DNA sequence or CpG island); optimised PCR buffers and deoxynucleotides; gel extraction kit; positive control primers; Ms-SNuPE primers for specific gene; reaction buffer (for the Ms-SNuPE reaction); and labelled nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

MSP (methylation-specific PCR) allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite converting all unmethylated, but not methylated cytosines to uracil, and subsequently amplified with primers specific for methylated versus unmethylated DNA.MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific gene (or bisulfite treated DNA sequence or CpG island), optimised PCR buffers and deoxynucleotides, and specific probes.

The MCA technique is a method that can be used to screen for altered methylation patterns in genomic DNA, and to isolate specific sequences associated with these changes (Toyota et al., Cancer Res. 59:2307-12, 1999). Briefly, restriction enzymes with different sensitivities to cytosine methylation in their recognition sites are used to digest genomic DNAs from primary tumours, cell lines, and normal tissues prior to arbitrarily primed PCR amplification. Fragments that show differential methylation are cloned and sequenced after resolving the PCR products on high-resolution polyacrylamide gels. The cloned fragments are then used as probes for Southern analysis to confirm differential methylation of these regions. Typical reagents (e.g., as might be found in a typical MCA-based kit) for MCA analysis may include, but are not limited to: PCR primers for arbitrary priming genomic DNA; PCR buffers and nucleotides, restriction enzymes and appropriate buffers; gene-hybridisation oligos or probes; control hybridisation oligos or probes.

The genomic sequences according to SEQ ID NO: 1 to SEQ ID NO: 56, and non-naturally occurring (e.g. chemically) treated variants thereof according to SEQ ID NO: 236 to SEQ ID NO: 347, were determined to have utility for the detection and/or classification of lung cell proliferative disorders.

In one embodiment the invention provides a method for detecting and/or for detecting and distinguishing between or among lung cell proliferative disorders in a subject. Said method comprises the following steps

i) contacting genomic DNA isolated from bodily fluids obtained from the subject with at least one reagent, or series of reagents that distinguishes between methylated and non-methylated CpG dinucleotides within at least one target region of the genomic DNA, wherein said contiguous nucleotides comprise at least one CpG dinucleotide sequence, and
ii) detecting, or detecting and distinguishing between or among lung cell proliferative disorders based on the methylated and non-methylated CpG dinucleotides.

Genomic DNA may be isolated by any means standard in the art, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants e.g. by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA. Body fluids are the preferred source of the DNA; particularly preferred are sputum, blood plasma, blood serum, whole blood, isolated blood cells and cells isolated from the blood.

The genomic DNA sample is then treated in such a manner that cytosine bases which are unmethylated at the 5′-position are converted to uracil, thymine, or another base which is dissimilar to cytosine in terms of hybridisation behaviour. This will be understood as “treatment” or “chemical treatment” herein.

The above described treatment of genomic DNA is preferably carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behaviour.

The treated DNA is then analysed in order to determine the methylation state of one or more target gene sequences (prior to the treatment) associated with the development of NSCLC. It is particularly preferred that the target region comprises, or hybridises under stringent conditions to at least 16 contiguous nucleotides of at least one gene or genomic sequence selected from the group consisting the genes and genomic sequences AKT2, BCL2, CDKN2A, ERBB4, FOS, GSTP1, HRAS, MDR1(ABCB1), RARB, ESR1, BCL2L1, PIK3CA, MAPK1, EREG, RASSF1, IGF1R, EGF, IGF1, STAT1, AREG, GP1BB, PTPRCAP(RPS6KB2), STK15(STK6), BTC, FOXF1, SHC1, STK12(AURKB), RASGRP2, ABCC4, RASGRP1, RASGRF1, MVP, ADAM17(TACE), ABCC12, PAK7 and NRG3. It is further preferred that the sequences of said genes as described in the accompanying sequence listing (see Table 3) are analysed. The method of analysis may be selected from those known in the art, including those listed herein. Particularly preferred are MethyLight™, MSP and the use of blocking oligonucleotides as will be described herein. It is further preferred that any oligonucleotides used in such analysis (including primers, blocking oligonucleotides and detection probes) should be reverse complementary, identical, or hybridise under stringent or highly stringent conditions to an at least 16-base-pair long segment of the base sequences of one or more of SEQ ID NO: 236 to SEQ ID NO: 347 and sequences complementary thereto.

Aberrant methylation, more preferably hypermethylation of one or more genes taken from the group consisting AKT2, BCL2, CDKN2A, ERBB4, FOS, GSTP1, HRAS, MDR1(ABCB1), RARB, ESR1, BCL2L1, PIK3CA, MAPK1, EREG, RASSF1, IGF1R, EGF, IGF1, STAT1, AREG, GP1BB, PTPRCAP(RPS6 KB2), STK15(STK6), BTC, FOXF1, SHC1, STK12(AURKB), RASGRP2, ABCC4, RASGRP1, RASGRF1, MVP, ADAM17(TACE), ABCC12, PAK7 and NRG3 or genomic sequences thereof as listed in Table 3 are associated with the presence of lung carcinoma. Analysis of one or a plurality of the sequences enables detecting, or detecting and distinguishing between or among lung cell proliferative disorders.

In one embodiment the method discloses the use of one or more genes and their promoter or regulatory elements selected from the group consisting of AKT2, BCL2, CDKN2A, ERBB4, FOS, GSTP1, HRAS, MDR1(ABCB1), RARB, ESR1, BCL2L1, PIK3CA, MAPK1, EREG, RASSF1, IGF1R, EGF, IGF1, STAT1, AREG, GP1BB, PTPRCAP(RPS6 KB2), STK15(STK6), BTC, FOXF1, SHC1, STK12(AURKB), RASGRP2, ABCC4, RASGRP1, RASGRF1, MVP, ADAM17(TACE), ABCC12, PAK7 and NRG3 as markers for the detection of NSCLC.

Said use of the genes and/or sequences may be enabled by means of any analysis of the expression of the gene, by means of mRNA expression analysis or protein expression analysis. However, in the most preferred embodiment of the invention, the detection of lung cell proliferative disorders is enabled by means of analysis of the methylation status of said genes or genomic sequences and their promoter or regulatory elements. Methods for the methylation analysis of genes are described herein.

Aberrant expression, more preferably under-expression of one or more genes taken from the group consisting AKT2, BCL2, CDKN2A, ERBB4, FOS, GSTP1, HRAS, MDR1(ABCB1), RARB, ESR1, BCL2L1, PIK3CA, MAPK1, EREG, RASSF1, IGF1R, EGF, IGF1, STAT1, AREG, GP1BB, PTPRCAP(RPS6 KB2), STK15(STK6), BTC, FOXF1, SHC1, STK12(AURKB), RASGRP2, ABCC4, RASGRP1, RASGRF1, MVP, ADAM17(TACE), ABCC12, PAK7 and NRG3 or genomic sequences thereof as listed in Table 3 are associated with the presence of lung carcinoma.

In one embodiment the method discloses the use of the gene IGF1 and/or its promoter or regulatory elements as a marker for the differentiation of lung squamous cell carcinoma from lung adenocarcinoma. Said use of the gene and/or sequences thereof may be enabled by means of any analysis of the expression of the gene, by means of mRNA expression analysis or protein expression analysis. However, in the most preferred embodiment of the invention, the detection of lung cell proliferative disorders is enabled by means of analysis of the methylation status of said genes or genomic sequences and their promoter or regulatory elements. Methods for the methylation analysis of genes are described herein.

In one embodiment the method discloses the use of one or more genes and their promoter or regulatory elements selected from the group consisting of IGF1, AREG and RASGRP1 as markers for the detection of lung squamous cell carcinoma. Said use of the genes and/or sequences may be enabled by means of any analysis of the expression of the gene, by means of mRNA expression analysis or protein expression analysis. However, in the most preferred embodiment of the invention, the detection of lung cell proliferative disorders is enabled by means of analysis of the methylation status of said genes or genomic sequences and their promoter or regulatory elements. Methods for the methylation analysis of genes are described herein.

In one embodiment the method discloses the use of one or more genes and their promoter or regulatory elements selected from the group consisting of AREG, GP1BB, FOXF1, RASGRP2 and NRG3 as markers for the detection of lung adenocarcinoma. Said use of the genes and/or sequences may be enabled by means of any analysis of the expression of the gene, by means of mRNA expression analysis or protein expression analysis. However, in the most preferred embodiment of the invention, the detection of lung cell proliferative disorders is enabled by means of analysis of the methylation status of said genes or genomic sequences and their promoter or regulatory elements. Methods for the methylation analysis of genes are described herein.

Aberrant levels of mRNA expression of the genes AKT2, BCL2, CDKN2A, ERBB4, FOS, GSTP1, HRAS, MDR1(ABCB1), RARB, ESR1, BCL2L1, PIK3CA, MAPK1, EREG, RASSF1, IGF1R, EGF, IGF1, STAT1, AREG, GP1BB, PTPRCAP(RPS6 KB2), STK15(STK6), BTC, FOXF1, SHC1, STK12(AURKB), RASGRP2, ABCC4, RASGRP1, RASGRF1, MVP, ADAM17(TACE), ABCC12, PAK7 and NRG3 are associated with lung cell proliferative disorders. Accordingly, increased or decreased levels of expression of said genes or sequences are associable with the development of lung cancers and other lung cell proliferative disorders.

To detect the presence of mRNA encoding a gene or genomic sequence in a detection system for lung cancer, a sample is obtained from a patient. The sample can be a tissue biopsy sample or a sample of blood, plasma, serum or the like. The sample may be treated to extract the nucleic acids contained therein. The resulting nucleic acid from the sample is subjected to gel electrophoresis or other separation techniques. Detection involves contacting the nucleic acids and in particular the mRNA of the sample with a DNA sequence serving as a probe to form hybrid duplexes. The stringency of hybridisation is determined by a number of factors during hybridisation and during the washing procedure, including temperature, ionic strength, length of time and concentration of formamide. These factors are outlined in, for example, Sambrook et al. (Molecular Cloning: A Laboratory Manual, 2d ed., 1989). Detection of the resulting duplex is usually accomplished by the use of labelled probes. Alternatively, the probe may be unlabeled, but may be detectable by specific binding with a ligand which is labelled, either directly or indirectly. Suitable labels and methods for labelling probes and ligands are known in the art, and include, for example, radioactive labels which may be incorporated by known methods (e.g., nick translation or kinasing), biotin, fluorescent groups, chemiluminescent groups (e.g., dioxetanes, particularly triggered dioxetanes), enzymes, antibodies, and the like.

In order to increase the sensitivity of the detection in a sample of mRNA transcribed from the gene or genomic sequence, the technique of reverse transcription/polymerisation chain reaction can be used to amplify cDNA transcribed from the mRNA. The method of reverse transcription/PCR is well known in the art (for example, see Watson and Fleming, supra).

The reverse transcription/PCR method can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end primer. Typically, the primer contains an oligo(dT) sequence. The cDNA thus produced is then amplified using the PCR method and gene specific primers. (Belyavsky et al, Nucl Acid Res 17:2919-2932, 1989; Krug and Berger, Methods in Enzymology, Academic Press, N.Y., Vol. 152, pp. 316-325, 1987 which are incorporated by reference)

The present invention may also be described in certain embodiments as a kit for use in detecting a lung cell proliferative disorder state through testing of a biological sample. A representative kit may comprise one or more nucleic acid segments that selectively hybridise to them, RNA and a container for each of the one or more nucleic acid segments. In certain embodiments the nucleic acid segments may be combined in a single tube. In further embodiments, the nucleic acid segments may also include a pair of primers for amplifying the target mRNA. Such kits may also include any buffers, solutions, solvents, enzymes, nucleotides, or other components for hybridisation, amplification or detection reactions. Preferred kit components include reagents for reverse transcription-PCR, in situ hybridisation, Northern analysis and/or ribonuclease protection assay (RPA).

The present invention further provides for methods to detect the presence of the polypeptide encoded by said genes or gene sequences in a sample obtained from a patient.

Aberrant levels of polypeptide expression of the polypeptides encoded by the genes, genomic sequences or genes regulated by genomic sequences of the group consisting AKT2, BCL2, CDKN2A, ERBB4, FOS, GSTP1, HRAS, MDR1(ABCB1), RARB, ESR1, BCL2L1, PIK3CA, MAPK1, EREG, RASSF1, IGF1R, EGF, IGF1, STAT1, AREG, GP1BB, PTPRCAP(RPS6 KB2), STK15(STK6), BTC, FOXF1, SHC1, STK12(AURKB), RASGRP2, ABCC4, RASGRP1, RASGRF1, MVP, ADAM17(TACE), ABCC12, PAK7 and NRG3 are associated with lung carcinoma. Accordingly over or under expression of said polypeptides are associable with the development of lung carcinoma and other lung cell proliferative disorders.

Any method known in the art for detecting proteins can be used. Such methods include, but are not limited to immunodiffusion, immunoelectrophoresis, immunochemical methods, binder-ligand assays, immunohistochemical techniques, agglutination and complement assays. (for example see Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labelled protein or derivative thereof.

Certain embodiments of the present invention comprise the use of antibodies specific to the polypeptide encoded by the genes or genomic sequences of the group consisting of AKT2, BCL2, CDKN2A, ERBB4, FOS, GSTP1, HRAS, MDR1(ABCB1), RARB, ESR1, BCL2L1, PIK3CA, MAPK1, EREG, RASSF1, IGF1R, EGF, IGF1, STAT1, AREG, GP1BB, PTPRCAP(RPS6 KB2), STK15(STK6), BTC, FOXF1, SHC1, STK12(AURKB), RASGRP2, ABCC4, RASGRP1, RASGRF1, MVP, ADAM17(TACE), ABCC12, PAK7 and NRG3.

Such antibodies may be useful for diagnostic and prognostic applications in detecting the disease state, by comparing a patient's levels of lung disease marker expression to expression of the same markers in normal individuals. In certain embodiments production of monoclonal or polyclonal antibodies can be induced by the use of the coded polypeptide as antigene. Such antibodies may in turn be used to detect expressed proteins as markers for human disease states. The levels of such proteins present in the peripheral blood or tissue sample of a patient may be quantified by conventional methods. Antibody-protein binding may be detected and quantified by a variety of means known in the art, such as labelling with fluorescent or radioactive ligands. The invention further comprises kits for performing the above-mentioned procedures, wherein such kits contain antibodies specific for the investigated polypeptides.

Numerous competitive and non-competitive protein binding immunoassays are well known in the art. Antibodies employed in such assays may be unlabeled, for example as used in agglutination tests, or labelled for use a wide variety of assay methods. Labels that can be used include radionuclides, enzymes, fluorescers, chemiluminescers, enzyme substrates or co-factors, enzyme inhibitors, particles, dyes and the like for use in radioimmunoassay (RIA), enzyme immunoassays, e.g., enzyme-linked immunosorbent assay (ELISA), fluorescent immunoassays and the like. Polyclonal or monoclonal antibodies or epitopes thereof can be made for use in immunoassays by any of a number of methods known in the art. One approach for preparing antibodies to a protein is the selection and preparation of an amino acid sequence of all or part of the protein, chemically synthesising the sequence and injecting it into an appropriate animal, usually a rabbit or a mouse (Milstein and Kohler Nature 256:495-497, 1975; Gulfre and Milstein, Methods in Enzymology: Immunochemical Techniques 73:1-46, Langone and Banatis eds., Academic Press, 1981 which are incorporated by reference). Methods for preparation of the polypeptides or epitopes thereof include, but are not limited to chemical synthesis, recombinant DNA techniques or isolation from biological samples.

In a further embodiment the present invention is based upon the analysis of methylation levels within two or more genes or genomic sequences taken from the group consisting of AKT2, BCL2, CDKN2A, ERBB4, FOS, GSTP1, HRAS, MDR1(ABCB1), RARB, ESR1, BCL2L1, PIK3CA, MAPK1, EREG, RASSF1, IGF1R, EGF, IGF1, STAT1, AREG, GP1BB, PTPRCAP(RPS6 KB2), STK15(STK6), BTC, FOXF1, SHC1, STK12(AURKB), RASGRP2, ABCC4, RASGRP1, RASGRF1, MVP, ADAM17(TACE), ABCC12, PAK7 and NRG3 and/or their regulatory sequences. It is further preferred that the sequences of said genes or genomic sequences are as according to SEQ ID NO: 1 to SEQ ID NO: 56.

Particular embodiments of the present invention provide a novel application of the analysis of methylation levels and/or patterns within said sequences that enables a detection and/or differentiation of lung cell proliferative disorders. Early detection and classification of lung cell proliferative disorders is linked with disease prognosis, and the disclosed method thereby enables the physician and patient to make better and more informed therapeutic decisions.

### Further Improvements

The present invention provides novel uses for genomic sequences selected from the group consisting of SEQ ID NO: 1 to SEQ ID NO: 56. Additional embodiments provide modified variants of SEQ ID NO: 1 to SEQ ID NO: 56, as well as oligonucleotides and/or PNA-oligomers for analysis of cytosine methylation patterns within the group consisting SEQ ID NO: 1 to SEQ ID NO: 56.

An objective of the invention comprises analysis of the methylation state of one or more CpG dinucleotides within at least one of the genomic sequences selected from the group consisting of SEQ ID NO: 1 to SEQ ID NO: 56 and sequences complementary thereto.

The disclosed invention provides treated nucleic acids, derived from genomic SEQ ID NO: 1 to SEQ ID NO: 56, wherein the treatment is suitable to convert at least one unmethylated cytosine base of the genomic DNA sequence to uracil or another base that is detectably dissimilar to cytosine in terms of hybridisation. The genomic sequences in question may comprise one, or more, consecutive or random methylated CpG positions. Said treatment preferably comprises use of a reagent selected from the group consisting of bisulfite, hydrogen sulfite, disulfite, and combinations thereof. In a preferred embodiment of the invention, the objective comprises analysis of a non-naturally occurring modified nucleic acid comprising a sequence of at least 16 contiguous nucleotide bases in length of a sequence selected from the group consisting of SEQ ID NO: 236 to SEQ ID NO: 347, wherein said sequence comprises at least one CpG, TpA or CpA dinucleotide and sequences complementary thereto. The sequences of SEQ ID NO: 236 to SEQ ID NO: 347 provide non-naturally occurring modified versions of the nucleic acid according to SEQ ID NO: 1 to SEQ ID NO: 56, wherein the modification of each genomic sequence results in the synthesis of a nucleic acid having a sequence that is unique and distinct from said genomic sequence as follows. For each sense strand genomic DNA, e.g., SEQ ID NO: 1, four converted versions are disclosed. A first version wherein “C” to “T,” but “CpG” remains “CpG” (i.e., corresponds to case where, for the genomic sequence, all “C” residues of CpG dinucleotide sequences are methylated and are thus not converted); a second version discloses the complement of the disclosed genomic DNA sequence (i.e. antisense strand), wherein “C” to “T,” but “CpG” remains “CpG” (i.e., corresponds to case where, for all “C” residues of CpG dinucleotide sequences are methylated and are thus not converted). The ‘upmethylated’ converted sequences of SEQ ID NO: 1 to SEQ ID NO: 56 correspond to SEQ ID NO: 236 to SEQ ID NO: 347. A third chemically converted version of each genomic sequences is provided, wherein “C” to “T” for all “C” residues, including those of “CpG” dinucleotide sequences (i.e., corresponds to case where, for the genomic sequences, all “C” residues of CpG dinucleotide sequences are unmethylated); a final chemically converted version of each sequence, discloses the complement of the disclosed genomic DNA sequence (i.e. antisense strand), wherein “C” to “T” for all “C” residues, including those of “CpG” dinucleotide sequences (i.e., corresponds to case where, for the complement (antisense strand) of each genomic sequence, all “C” residues of CpG dinucleotide sequences are unmethylated). The ‘downmethylated’ converted sequences of SEQ ID NO: 1 to SEQ ID NO: 56 correspond to SEQ ID NO: 236 to SEQ ID NO: 347.

In an alternative preferred embodiment, such analysis comprises the use of an oligonucleotide or oligomer for detecting the cytosine methylation state within genomic or treated (chemically modified) DNA, according to SEQ ID NO: 236 to SEQ ID NO: 347. Said oligonucleotide or oligomer comprising a nucleic acid sequence having a length of at least nine (9) nucleotides which hybridizes, under moderately stringent or stringent conditions (as defined herein above), to a treated nucleic acid sequence according to SEQ ID NO: 236 to SEQ ID NO: 347 and/or sequences complementary thereto, or to a genomic sequence according to SEQ ID NO: 1 to SEQ ID NO: 56 and/or sequences complementary thereto. Particularly preferred is a nucleic acid molecule that hybridize under moderately stringent and/or stringent hybridization conditions to all or a portion of the sequences SEQ ID NO: 236 to SEQ ID NO: 347 but not SEQ ID NO: 1 to SEQ ID NO: 56 or other human genomic DNA.

Thus, the present invention includes nucleic acid molecules (e.g., oligonucleotides and peptide nucleic acid (PNA) molecules (PNA-oligomers)) that hybridise under moderately stringent and/or stringent hybridisation conditions to all or a portion of the sequences SEQ ID NO: 1 to SEQ ID NO: 347, or to the complements thereof. The hybridising portion of the hybridizing nucleic acids is typically at least 9, 15, 20, 25, 30 or 35 nucleotides in length. However, longer molecules have inventive utility, and are thus within the scope of the present invention. Particularly preferred is a nucleic acid molecule that hybridize under moderately stringent and/or stringent hybridization conditions to all or a portion of the sequences SEQ ID NO: 236 to SEQ ID NO: 347 but not SEQ ID NO: 1 to SEQ ID NO: 56 or other human genomic DNA.

Preferably, the hybridising portion of the inventive hybridising nucleic acids is at least 95%, or at least 98%, or 100% identical to the sequence, or to a portion thereof of SEQ ID NO: 1 to SEQ ID NO: 347, or to the complements thereof. Particularly preferred is a nucleic acid molecule that hybridize under moderately stringent and/or stringent hybridization conditions to all or a portion of the sequences SEQ ID NO: 236 to SEQ ID NO: 347 but not SEQ ID NO: 1 to SEQ ID NO: 56 or other human genomic DNA.

Hybridising nucleic acids of the type described herein can be used, for example, as a primer (e.g., a PCR primer), or a diagnostic and/or prognostic probe or primer. Preferably, hybridisation of the oligonucleotide probe to a nucleic acid sample is performed under stringent conditions and the probe is 100% identical to the target sequence. Nucleic acid duplex or hybrid stability is expressed as the melting temperature or Tm, which is the temperature at which a probe dissociates from a target DNA. This melting temperature is used to define the required stringency conditions.

For target sequences that are related and substantially identical to the corresponding sequence of SEQ ID NO: 1 to SEQ ID NO: 56 (such as allelic variants and SNPs), rather than identical, it is useful to first establish the lowest temperature at which only homologous hybridisation occurs with a particular concentration of salt (e.g., SSC or SSPE). Then, assuming that 1% mismatching results in a 1° C. decrease in the Tm, the temperature of the final wash in the hybridisation reaction is reduced accordingly (for example, if sequences having >95% identity with the probe are sought, the final wash temperature is decreased by 5° C.). In practice, the change in Tm can be between 0.5° C. and 1.5° C. per 1% mismatch.

Examples of inventive oligonucleotides of length X (in nucleotides), as indicated by polynucleotide positions with reference to, e.g., SEQ ID NO: 1, include those corresponding to sets (sense and antisense sets) of consecutively overlapping oligonucleotides of length X, where the oligonucleotides within each consecutively overlapping set (corresponding to a given X value) are defined as the finite set of Z oligonucleotides from nucleotide positions:

n to (n+(X−1));
where n=1, 2, 3, . . . (Y−(X−1));
where Y equals the length (nucleotides or base pairs) of SEQ ID NO: 236 (6197);
where X equals the common length (in nucleotides) of each oligonucleotide in the set (e.g., X=20 for a set of consecutively overlapping 20-mers); and
where the number (Z) of consecutively overlapping oligomers of length X for a given SEQ ID NO of length Y is equal to Y−(X−1). For example Z=6197−19=6178 for either sense or antisense sets of SEQ ID NO: 236, where X=20.

Preferably, the set is limited to those oligomers that comprise at least one CpG, TpG or CpA dinucleotide.

Examples of inventive 20-mer oligonucleotides include the following set of oligomers (and the antisense set complementary thereto), indicated by polynucleotide positions with reference to SEQ ID NO: 236: 1-20, 2-21, 3-22, 4-23, 5-24, . . . 6178-6197.

Preferably, the set is limited to those oligomers that comprise at least one CpG, TpG or CpA dinucleotide.

Likewise, examples of inventive 25-mer oligonucleotides include the following set of oligomers (and the antisense set complementary thereto), indicated by polynucleotide positions with reference to SEQ ID NO: 236: 1-25, 2-26, 3-27, 4-28, 5-29, . . . 6172-6197.

Preferably, the set is limited to those oligomers that comprise at least one CpG, TpG or CpA dinucleotide.

The present invention encompasses, for each of SEQ ID NO: 1 to SEQ ID NO: 347 (sense and antisense), multiple consecutively overlapping sets of oligonucleotides or modified oligonucleotides of length X, where, e.g., X=9, 10, 17, 20, 22, 23, 25, 27, 30 or 35 nucleotides.

The oligonucleotides or oligomers according to the present invention constitute effective tools useful to ascertain genetic and epigenetic parameters of the genomic sequence corresponding to SEQ ID NO: 1 to SEQ ID NO: 56. Preferred sets of such oligonucleotides or modified oligonucleotides of length X are those consecutively overlapping sets of oligomers corresponding to SEQ ID NO: 1 to SEQ ID NO: 347 (and to the complements thereof). Preferably, said oligomers comprise at least one CpG, TpG or CpA dinucleotide.

Particularly preferred oligonucleotides or oligomers according to the present invention are those in which the cytosine of the CpG dinucleotide (or of the corresponding converted TpG or CpA dinucleotide) sequences is within the middle third of the oligonucleotide; that is, where the oligonucleotide is, for example, 13 bases in length, the CpG, TpG or CpA dinucleotide is positioned within the fifth to ninth nucleotide from the 5′-end.

The oligonucleotides of the invention can also be modified by chemically linking the oligonucleotide to one or more moieties or conjugates to enhance the activity, stability or detection of the oligonucleotide. Such moieties or conjugates include chromophores, fluorophores, lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773. The probes may also exist in the form of a PNA (peptide nucleic acid) which has particularly preferred pairing properties. Thus, the oligonucleotide may include other appended groups such as peptides, and may include hybridisation-triggered cleavage agents (Krol et al., BioTechniques 6:958-976, 1988) or intercalating agents (Zon, Pharm. Res. 5:539-549, 1988). To this end, the oligonucleotide may be conjugated to another molecule, e.g., a chromophore, fluorophor, peptide, hybridisation-triggered cross-linking agent, transport agent, hybridisation-triggered cleavage agent, etc.

The oligonucleotide may also comprise at least one art-recognised modified sugar and/or base moiety, or may comprise a modified backbone or non-natural internucleoside linkage.

The oligonucleotides or oligomers according to particular embodiments of the present invention are typically used in ‘sets,’ which contain at least one oligomer for analysis of each of the CpG dinucleotides of genomic sequences SEQ ID NO: 1 to SEQ ID NO: 56 and sequences complementary thereto, or to the corresponding CpG, TpG or CpA dinucleotide within a sequence of the treated nucleic acids according to SEQ ID NO: 236 to SEQ ID NO: 347 and sequences complementary thereto. However, it is anticipated that for economic or other factors it may be preferable to analyse a limited selection of the CpG dinucleotides within said sequences, and the content of the set of oligonucleotides is altered accordingly.

Therefore, in particular embodiments, the present invention provides a set of at least two (2) (oligonucleotides and/or PNA-oligomers) useful for detecting the cytosine methylation state in treated genomic DNA (SEQ ID NO: 236 to SEQ ID NO: 347), or in genomic DNA (SEQ ID NO: 1 to SEQ ID NO: 56 and sequences complementary thereto). These probes enable diagnosis, and/or classification of genetic and epigenetic parameters of lung cell proliferative disorders. The set of oligomers may also be used for detecting single nucleotide polymorphisms (SNPs) in treated genomic DNA (SEQ ID NO: 236 to SEQ ID NO: 347), or in genomic DNA (SEQ ID NO: 1 to SEQ ID NO: 56 and sequences complementary thereto).

In preferred embodiments, at least one, and more preferably all members of a set of oligonucleotides is bound to a solid phase.

In further embodiments, the present invention provides a set of at least two (2) oligonucleotides that are used as ‘primer’ oligonucleotides for amplifying DNA sequences of one of SEQ ID NO: 1 to SEQ ID NO: 347 and sequences complementary thereto, or segments thereof.

It is anticipated that the oligonucleotides may constitute all or part of an “array” or “DNA chip” (i.e., an arrangement of different oligonucleotides and/or PNA-oligomers bound to a solid phase). Such an array of different oligonucleotide- and/or PNA-oligomer sequences can be characterised, for example, in that it is arranged on the solid phase in the form of a rectangular or hexagonal lattice. The solid-phase surface may be composed of silicon, glass, polystyrene, aluminium, steel, iron, copper, nickel, silver, or gold. Nitrocellulose as well as plastics such as nylon, which can exist in the form of pellets or also as resin matrices, may also be used. An overview of the Prior Art in oligomer array manufacturing can be gathered from a special edition of Nature Genetics (Nature Genetics Supplement, Volume 21, January 1999, and from the literature cited therein). Fluorescently labelled probes are often used for the scanning of immobilised DNA arrays. The simple attachment of Cy3 and Cy5 dyes to the 5′-OH of the specific probe are particularly suitable for fluorescence labels. The detection of the fluorescence of the hybridised probes may be carried out, for example, via a confocal microscope. Cy3 and Cy5 dyes, besides many others, are commercially available.

It is also anticipated that the oligonucleotides, or particular sequences thereof, may constitute all or part of an “virtual array” wherein the oligonucleotides, or particular sequences thereof, are used, for example, as ‘specifiers’ as part of, or in combination with a diverse population of unique labelled probes to analyse a complex mixture of analytes. Such a method, for example is described in US 2003/0013091 (U.S. Ser. No. 09/898,743, published 16 Jan. 2003). In such methods, enough labels are generated so that each nucleic acid in the complex mixture (i.e., each analyte) can be uniquely bound by a unique label and thus detected (each label is directly counted, resulting in a digital read-out of each molecular species in the mixture).

It is particularly preferred that the oligomers according to the invention are utilised for at least one of: detection of; detection and differentiation between or among subclasses of; diagnosis of; and monitoring of lung cell proliferative disorders. This is enabled by use of said sets for the differentiation and/or detection of the tissue types according to table 4-11. Particularly preferred are those sets of oligomer that comprise at least two oligonucleotides selected from one of the following sets of oligonucleotides.

In one embodiment of the method, lung cancer tissue is detected. This is achieved by analysis of the methylation status of at least one target sequence comprising, or hybridising under stringent conditions to at least 16 contiguous nucleotides of a gene (or sequence thereof according to Table 3) selected from the group consisting AKT2, BCL2, CDKN2A, ERBB4, FOS, GSTP1, HRAS, MDR1(ABCB1), RARB, ESR1, BCL2L1, PIK3CA, MAPK1, EREG, RASSF1, IGF1R, EGF, IGF1, STAT1, AREG, GP1BB, PTPRCAP(RPS6 KB2), STK15(STK6), BTC, FOXF1, SHC1, STK12(AURKB), RASGRP2, ABCC4, RASGRP1, RASGRF1, MVP, ADAM17(TACE), ABCC12, PAK7 and NRG3 and complements thereof. This is preferably achieved by use of a set consisting of at least one oligonucleotide, and more preferably at least two selected from one of the groups consisting of SEQ ID NOS: 1612-1755.

In one embodiment of the method, lung squamous cell carcinoma and lung adenocarcinoma are differentiated from one another. This is achieved by analysis of the methylation status of at least one target sequence comprising, or hybridising under stringent conditions to at least 16 contiguous nucleotides of the gene (or sequence thereof according to Table 3) IGF1 and complements thereof. This is preferably achieved by use of a set consisting of at least one oligonucleotide, and more preferably at least two selected from one of the groups consisting of SEQ ID NOS: 1703-1707.

In one embodiment of the method, lung squamous cell carcinoma is detected. This is achieved by analysis of the methylation status of at least one target sequence comprising, or hybridising under stringent conditions to at least 16 contiguous nucleotides of a gene (or sequence thereof according to Table 3) selected from the group consisting IGF1, AREG and RASGRP1 and complements thereof. This is preferably achieved by use of a set consisting of at least one oligonucleotide, and more preferably at least two selected from one of the groups consisting of SEQ ID NOS: 1703-1707, 1710-1713, 1753.

In one embodiment of the method, lung adenocarcinoma is detected. This is achieved by analysis of the methylation status of at least one target sequence comprising, or hybridising under stringent conditions to at least 16 contiguous nucleotides of a gene (or sequence thereof according to Table 3) selected from the group consisting AREG, GP1BB, FOXF1, RASGRP2 and NRG3 and complements thereof. This is preferably achieved by use of a set consisting of at least one oligonucleotide, and more preferably at least two selected from one of the groups consisting of SEQ ID NOS: 1661, 1663, 1710-1713, 1718, 1728-1732, 1745-1748.

The present invention further provides a method for ascertaining genetic and/or epigenetic parameters of the genomic sequences according to SEQ ID NO: 1 to SEQ ID NO: 56 within a subject by analysing cytosine methylation and single nucleotide polymorphisms. Said method comprising contacting a nucleic acid comprising one or more of SEQ ID NO: 1 to SEQ ID NO: 56 in a biological sample obtained from said subject with at least one reagent or a series of reagents, wherein said reagent or series of reagents, distinguishes between methylated and non-methylated CpG dinucleotides within the target nucleic acid.

Preferably, said method comprises the following steps: In the first step, a sample of the tissue to be analysed is obtained. The source may be any suitable source, such as sputum, cell lines, histological slides, biopsies, paraffin-embedded tissue, bodily fluids, blood plasma, blood serum, whole blood, isolated blood cells, cells isolated from the blood and all possible combinations thereof.

The genomic DNA is then isolated from the sample. Genomic DNA may be isolated by any means standard in the art, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants e.g. by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA.

Once the nucleic acids have been extracted, the genomic double stranded DNA is used in the analysis.

In the second step of the method, the genomic DNA sample is treated in such a manner that cytosine bases which are unmethylated at the 5′-position are converted to uracil, thymine, or another base which is dissimilar to cytosine in terms of hybridisation behaviour. This will be understood as ‘pretreatment’ or ‘treatment’ herein.

The above-described treatment of genomic DNA is preferably carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behaviour.

In the third step of the method, fragments of the treated DNA are amplified, using sets of primer oligonucleotides according to the present invention, and an amplification enzyme. The amplification of several DNA segments can be carried out simultaneously in one and the same reaction vessel. Typically, the amplification is carried out using a polymerase chain reaction (PCR). The set of primer oligonucleotides includes at least two oligonucleotides whose sequences are each reverse complementary, identical, or hybridise under stringent or highly stringent conditions to an at least 16-base-pair long segment of the base sequences of one of SEQ ID NO: 236 to SEQ ID NO: 347 and sequences complementary thereto.

In an alternate embodiment of the method, the methylation status of preselected CpG positions within the nucleic acid sequences comprising one or more of SEQ ID NO: 1 to SEQ ID NO: 56 may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primers pairs contain at least one primer which hybridises to a bisulfite treated CpG dinucleotide. Therefore, the sequence of said primers comprises at least one CpG dinucleotide. MSP primers specific for non-methylated DNA contain a “T’ at the position of the C position in the CpG. Preferably, therefore, the base sequence of said primers is required to comprise a sequence having a length of at least 9 nucleotides which hybridises to a treated nucleic acid sequence according to one of SEQ ID NO: 236 to SEQ ID NO: 347 and sequences complementary thereto, wherein the base sequence of said oligomers comprises at least one CpG dinucleotide.

A further preferred embodiment of the method comprises the use of blocker oligonucleotides. The use of such blocker oligonucleotides has been described by Yu et al., BioTechniques 23:714-720, 1997. Blocking probe oligonucleotides are hybridised to the bisulfite treated nucleic acid concurrently with the PCR primers. PCR amplification of the nucleic acid is terminated at the 5′ position of the blocking probe, such that amplification of a nucleic acid is suppressed where the complementary sequence to the blocking probe is present. The probes may be designed to hybridise to the bisulfite treated nucleic acid in a methylation status specific manner. For example, for detection of methylated nucleic acids within a population of unmethylated nucleic acids, suppression of the amplification of nucleic acids which are unmethylated at the position in question would be carried out by the use of blocking probes comprising a ‘CpA’ or ‘TpA’ at the position in question, as opposed to a ‘CpG’ if the suppression of amplification of methylated nucleic acids is desired.

For PCR methods using blocker oligonucleotides, efficient disruption of polymerase-mediated amplification requires that blocker oligonucleotides not be elongated by the polymerase. Preferably, this is achieved through the use of blockers that are 3′-deoxyoligonucleotides, or oligonucleotides derivatized at the 3′ position with other than a “free” hydroxyl group. For example, 3′-O-acetyl oligonucleotides are representative of a preferred class of blocker molecule.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having, for example, thioate bridges at the 5′-termini thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will not extend the primer toward, and through (in the 5′-3′ direction) the blocker—a process that normally results in degradation of the hybridised blocker oligonucleotide.

A particularly preferred blocker/PCR embodiment, for purposes of the present invention and as implemented herein, comprises the use of peptide nucleic acid (PNA) oligomers as blocking oligonucleotides. Such PNA blocker oligomers are ideally suited, because they are neither decomposed nor extended by the polymerase.

Preferably, therefore, the base sequence of said blocking oligonucleotides is required to comprise a sequence having a length of at least 9 nucleotides which hybridises to a treated nucleic acid sequence according to one of SEQ ID NO: 236 to SEQ ID NO: 347 and sequences complementary thereto, wherein the base sequence of said oligonucleotides comprises at least one CpG, TpG or CpA dinucleotide.

The fragments obtained by means of the amplification can carry a directly or indirectly detectable label. Preferred are labels in the form of fluorescence labels, radionuclides, or detachable molecule fragments having a typical mass which can be detected in a mass spectrometer. Where said labels are mass labels, it is preferred that the labelled amplificates have a single positive or negative net charge, allowing for better detectability in the mass spectrometer. The detection may be carried out and visualised by means of, e.g., matrix assisted laser desorption/ionisation mass spectrometry (MALDI) or using electron spray mass spectrometry (ESI).

Matrix Assisted Laser Desorption/Ionisation Mass Spectrometry (MALDI-TOF) is a very efficient development for the analysis of biomolecules (Karas and Hillenkamp, Anal Chem., 60:2299-301, 1988). An analyte is embedded in a light-absorbing matrix. The matrix is evaporated by a short laser pulse thus transporting the analyte molecule into the vapour phase in an unfragmented manner. The analyte is ionised by collisions with matrix molecules. An applied voltage accelerates the ions into a field-free flight tube. Due to their different masses, the ions are accelerated at different rates. Smaller ions reach the detector sooner than bigger ones. MALDI-TOF spectrometry is well suited to the analysis of peptides and proteins. The analysis of nucleic acids is somewhat more difficult (Gut and Beck, Current Innovations and Future Trends, 1:147-57, 1995). The sensitivity with respect to nucleic acid analysis is approximately 100-times less than for peptides, and decreases disproportionally with increasing fragment size. Moreover, for nucleic acids having a multiply negatively charged backbone, the ionisation process via the matrix is considerably less efficient. In MALDI-TOF spectrometry, the selection of the matrix plays an eminently important role. For desorption of peptides, several very efficient matrixes have been found which produce a very fine crystallisation. There are now several responsive matrixes for DNA, however, the difference in sensitivity between peptides and nucleic acids has not been reduced. This difference in sensitivity can be reduced, however, by chemically modifying the DNA in such a manner that it becomes more similar to a peptide. For example, phosphorothioate nucleic acids, in which the usual phosphates of the backbone are substituted with thiophosphates, can be converted into a charge-neutral DNA using simple alkylation chemistry (Gut and Beck, Nucleic Acids Res. 23: 1367-73, 1995). The coupling of a charge tag to this modified DNA results in an increase in MALDI-TOF sensitivity to the same level as that found for peptides. A further advantage of charge tagging is the increased stability of the analysis against impurities, which makes the detection of unmodified substrates considerably more difficult.

In the fourth step of the method, the amplificates obtained during the third step of the method are analysed in order to ascertain the methylation status of the CpG dinucleotides prior to the treatment.

In embodiments where the amplificates were obtained by means of MSP amplification, the presence or absence of an amplificate is in itself indicative of the methylation state of the CpG positions covered by the primer, according to the base sequences of said primer.

Amplificates obtained by means of both standard and methylation specific PCR may be further analysed by means of hybridisation-based methods such as, but not limited to, array technology and probe based technologies as well as by means of techniques such as sequencing and template directed extension.

In one embodiment of the method, the amplificates synthesised in step three are subsequently hybridised to an array or a set of oligonucleotides and/or PNA probes. In this context, the hybridisation takes place in the following manner: the set of probes used during the hybridisation is preferably composed of at least 2 oligonucleotides or PNA-oligomers; in the process, the amplificates serve as probes which hybridise to oligonucleotides previously bonded to a solid phase; the non-hybridised fragments are subsequently removed; said oligonucleotides contain at least one base sequence having a length of at least 9 nucleotides which is reverse complementary or identical to a segment of the base sequences specified in the present Sequence Listing; and the segment comprises at least one CpG, TpG or CpA dinucleotide.

In a preferred embodiment, said dinucleotide is present in the central third of the oligomer. For example, wherein the oligomer comprises one CpG dinucleotide, said dinucleotide is preferably the fifth to ninth nucleotide from the 5′-end of a 13-mer. One oligonucleotide exists for the analysis of each CpG dinucleotide within the sequence according to SEQ ID NO: 1 to SEQ ID NO: 56, and the equivalent positions within SEQ ID NO: 236 to SEQ ID NO: 347. Said oligonucleotides may also be present in the form of peptide nucleic acids. The non-hybridised amplificates are then removed. The hybridised amplificates are then detected. In this context, it is preferred that labels attached to the amplificates are identifiable at each position of the solid phase at which an oligonucleotide sequence is located.

In yet a further embodiment of the method, the genomic methylation status of the CpG positions may be ascertained by means of oligonucleotide probes that are hybridised to the bisulfite treated DNA concurrently with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996; also see U.S. Pat. No. 6,331,393) employing a dual-labelled fluorescent oligonucleotide probe (TaqMan™ PCR, using an ABI Prism 7700 Sequence Detection System, Perkin Elmer Applied Biosystems, Foster City, Calif.). The TaqMan™ PCR reaction employs the use of a non-extendible interrogating oligonucleotide, called a TaqMan™ probe, which, in preferred embodiments, is designed to hybridise to a GpC-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulfite treatment, it is required that the probe be methylation specific, as described in U.S. Pat. No. 6,331,393, (hereby incorporated by reference in its entirety) also known as the MethylLight™ assay. Variations on the TaqMan™ detection methodology that are also suitable for use with the described invention include the use of dual-probe technology (Lightcycler™) or fluorescent amplification primers (Sunrise™ technology). Both these techniques may be adapted in a manner suitable for use with bisulfite treated DNA, and moreover for methylation analysis within CpG dinucleotides.

A further suitable method for the use of probe oligonucleotides for the assessment of methylation by analysis of bisulfite treated nucleic acids In a further preferred embodiment of the method, the fifth step of the method comprises the use of template-directed oligonucleotide extension, such as MS-SNuPE as described by Gonzalgo and Jones, Nucleic Acids Res. 25:2529-2531, 1997.

In yet a further embodiment of the method, the fourth step of the method comprises sequencing and subsequent sequence analysis of the amplificate generated in the third step of the method (Sanger F., et al., Proc Natl Acad Sci USA 74:5463-5467, 1977).

In the most preferred embodiment of the method the genomic nucleic acids are isolated and treated according to the first three steps of the method outlined above, namely:

a) obtaining, from a subject, a biological sample having subject genomic DNA;
b) extracting or otherwise isolating the genomic DNA;
c) treating the genomic DNA of b), or a fragment thereof, with one or more reagents to convert cytosine bases that are unmethylated in the 5-position thereof to uracil or to another base that is detectably dissimilar to cytosine in terms of hybridisation properties; and wherein
d) amplifying subsequent to treatment in c) is carried out in a methylation specific manner, namely by use of methylation specific primers or blocking oligonucleotides, and further wherein
e) detecting of the amplificates is carried out by means of a real-time detection probe, as described above.

Preferably, where the subsequent amplification of d) is carried out by means of methylation specific primers, as described above, said methylation specific primers comprise a sequence having a length of at least 9 nucleotides which hybridises to a treated nucleic acid sequence according to one of SEQ ID NO: 236 to SEQ ID NO: 347 and sequences complementary thereto, wherein the base sequence of said oligomers comprises at least one CpG dinucleotide.

In an alternative most preferred embodiment of the method, the subsequent amplification of d) is carried out in the presence of blocking oligonucleotides, as described above. Said blocking oligonucleotides comprising a sequence having a length of at least 9 nucleotides which hybridises to a treated nucleic acid sequence according to one of SEQ ID NO: 236 to SEQ ID NO: 347 and sequences complementary thereto, wherein the base sequence of said oligomers comprises at least one CpG, TpG or CpA dinucleotide. Step e) of the method, namely the detection of the specific amplificates indicative of the methylation status of one or more CpG positions according to SEQ ID NO: Ito SEQ ID NO: 56 is carried out by means of real-time detection methods as described above.

In the final step of the method the presence, absence or molecular classification (differentiation between lung squamous cell carcinoma and lung adenocarcinoma) is determined. Preferably, the correlation of the methylation status of the marker CpG positions with the presence, absence or molecular classification of lung cell proliferative disorders is done substantially without human intervention. For the diagnosis of NSCLC hypermethylation of a gene or its promoter or regulatory regions is indicative of the presence of NSCLC, with the exception of the genes AREG, IGF1R, EGF, MAPK1, BCL2 and PTPRCAP/RPS6 KB2 in which cases hypomethylation is indicative of the presence of NSCLC. For the molecular classification (differentiation) of lung squamous cell carcinoma and lung adenocarcinoma the genes PIK3R1, BCL2L1 and IGF1 are hypermethylated in lung squamous cell carcinoma as relative to lung adenocarcinoma and the genes SRC and RASGRP2 are hypomethylated in lung squamous cell carcinoma as relative to lung adenocarcinoma.

It is particularly preferred that the classification of the sample is carried out by algorithmic means. The development of algorithmic methods for the classification of a sample based on the methylation status of the CpG positions within the panel are demonstrated in the examples.

In one embodiment machine learning predictors are trained on the methylation patterns at the investigated CpG sites of the samples with known status. A selection of the CpG positions which are discriminative for the machine learning predictor are used in the panel. In a particularly preferred embodiment of the method, both methods are combined; that is, the machine learning classifier is trained only on the selected CpG positions that are significantly differentially methylated between the classes according to the statistical analysis.

Additional embodiments of the invention provide a method for the analysis of the methylation status of genomic DNA according to the invention (SEQ ID NO: 1 to SEQ ID NO: 56, and complements thereof) without the need for pretreatment.

In the first step of such additional embodiments, the genomic DNA sample is isolated from tissue or cellular sources. Preferably, such sources include cell lines, histological slides, body fluids, or tissue embedded in paraffin. In the second step, the genomic DNA is extracted. Extraction may be by means that are standard to one skilled in the art, including but not limited to the use of detergent lysates, sonification and vortexing with glass beads. Once the nucleic acids have been extracted, the genomic double-stranded DNA is used in the analysis. In a preferred embodiment, the DNA may be cleaved prior to the treatment, and this may be by any means standard in the state of the art, in particular with methylation-sensitive restriction endonucleases.

In the third step, the DNA is then digested with one or more methylation sensitive restriction enzymes. The digestion is carried out such that hydrolysis of the DNA at the restriction site is informative of the methylation status of a specific CpG dinucleotide.

In the fourth step, which is optional but a preferred embodiment, the restriction fragments are amplified. This is preferably carried out using a polymerase chain reaction, and said amplificates may carry suitable detectable labels as discussed above, namely fluorophore labels, radionuclides and mass labels.

In the fifth step the amplificates are detected. The detection may be by any means standard in the art, for example, but not limited to, gel electrophoresis analysis, hybridisation analysis, incorporation of detectable tags within the PCR products, DNA array analysis, MALDI or ESI analysis.

Subsequent to the determination of the methylation state of the genomic nucleic acids the presence, absence or subclass of lung cell proliferative disorder is deduced based upon the methylation state of at least one CpG dinucleotide sequence of SEQ ID NO: 1 to SEQ ID NO: 56, or an average, or a value reflecting an average methylation state of a plurality of CpG dinucleotide sequences of SEQ ID NO: 1 to SEQ ID NO: 56.

Diagnostic Assays for lung cell proliferative disorders—The present invention enables diagnosis of events which are disadvantageous to patients or individuals in which important genetic and/or epigenetic parameters within one or more of SEQ ID NO: 1 to SEQ ID NO: 56 may be used as markers. More specifically, the present invention enables the screening of at-risk populations (e.g. smokers) for the early detection of lung cancers. Further embodiments of the method may also be used as alternatives to cytological screening for the classification of lung carcinomas.

Specifically, the present invention provides for diagnostic cancer assays based on measurement of differential methylation of one or more CpG dinucleotide sequences of SEQ ID NO: 1 to SEQ ID NO: 56, or of subregions thereof that comprise such a CpG dinucleotide sequence. Typically, such assays involve obtaining a tissue sample from a test tissue, performing an assay to measure the methylation status of at least one of one or more CpG dinucleotide sequences of SEQ ID NO: 1 to SEQ ID NO: 56 derived from the tissue sample, relative to a control sample, or a known standard and making a diagnosis or prognosis based thereon.

In particular preferred embodiments, inventive oligomers are used to assess the CpG dinucleotide methylation status, such as those based on SEQ ID NO: 1 to SEQ ID NO: 347, or arrays thereof, as well as in kits based thereon and useful for the diagnosis and/or classification of lung cell proliferative disorders.

Kits—Moreover, an additional aspect of the present invention is a kit comprising, for example: a bisulfite-containing reagent; a set of primer oligonucleotides containing at least two oligonucleotides whose sequences in each case correspond, are complementary, or hybridise under stringent or highly stringent conditions to a 16-base long segment of the sequences SEQ ID NO: 1 to SEQ ID NO: 347; oligonucleotides and/or PNA-oligomers; as well as instructions for carrying out and evaluating the described method. In a further preferred embodiment, said kit may further comprise standard reagents for performing a CpG position-specific methylation analysis, wherein said analysis comprises one or more of the following techniques: MS-SNuPE, MSP, MethyLight™, HeavyMethyl™, COBRA, and nucleic acid sequencing. However, a kit along the lines of the present invention can also contain only part of the aforementioned components. It is particularly preferred that the base sequence of said at least two oligonucleotides in each case correspond, are complementary, or hybridise under stringent or highly stringent conditions to a 16-base long segment of the sequences SEQ ID NO: 236 to SEQ ID NO: 347. It is further preferred that the base sequence of said oligomers comprises at least one CpG, CpA or TpG dinucleotide.

It is particularly preferred that the kit according to the present invention further comprises instructions for determining the absence or presence of a lung cell proliferative disorder or characteristics thereof.

While the present invention has been described with specificity in accordance with certain of its preferred embodiments, the following examples serve only to illustrate the invention and are not intended to limit the invention within the principles and scope of the broadest interpretations and equivalent configurations thereof.

### EXAMPLE 1

### Microarray Analysis

### Samples

To evaluate marker candidates a significant number of patient and control samples was analysed using the applicant's proprietary methylation sensitive Microarray technology. For the Microarray study two gene panels were analysed on a collection of 48 matched pairs of samples from a commercial supplier, each sample consisting of diseased tissue and normal adjacent tissue (hereinafter also referred to as NAT). An overview of patient samples collected for the microarray study is provided in Table 12.

### Gene Selection

An initial selection of 88 candidate marker genes were identified.

### DNA Extraction

Samples were received from a commercial supplier as 10 mm paraffin embedded tissue sections. DNA from tissue samples were isolated using the applicant's proprietary techniques. In brief, samples were de-paraffinated and lysed followed by bisulfite treatment and purification of the converted DNA.

### PCR Establishment and Multiplex PCR Optimisation

To amplify all gene fragments, PCR assays were designed to match bisulfite treated DNA and to allow amplification independent of the methylation status of the respective fragment. A standardised primer design workflow optimised by the applicant for bisulfite treated DNA was employed. Primers are listed in table 1.

To allow efficient amplification, individual PCR assays were combined into multiplex PCR (mPCR) assays usually combining up to 8 primer pairs into one mPCR assay. The performance of each mPCR was evaluated on
bisulfite converted DNA from pooled samples according to Table 12
bisulfite converted standard DNA from human blood (Promega G3041) as a positive control
a water control with no DNA template to show absence of contaminations and primer dimer formation.

Multiplex PCR products were analysed by agarose gel electrophoresis and fragment analysis on an ALF express II DNA Analysis System (Amersham Biosciences). The best performing combination of multiplex PCR sets were chosen.

### Oligonucleotide Probe Design and Selection.

Oligonucleotide probes were designed for accessible CpG positions within the amplificates that only matched the bisulfite converted DNA fragments. This enables the exclusion of signals arising from incomplete bisulfite conversion.

To estimate background hybridisation negative control oligonucleotides were designed, that matched none of the amplificates of a microarray amplificate set. Further positive control oligonucleotides and matching spiking oligonucleotides were designed. The labelled spiking oligonucleotides are added to the hybridisation solution and bind to the positive control oligonucleotides spotted at several positions on the microarray. This positive control system allows to estimate hybridisation signal distribution over one microarray (intrachip variability) and over the whole set of microarrays (interchip variability). Microarray oligonucleotides with 5′ C6 amino modifications were supplied by MWG (Ebersberg, Germany). Spiking oligonucleotides were supplied with a 5′-Cy5 fluorescent label. Oligonucleotide probes are listed in table 2.

### Bisulfite Treatment and Multiplex PCR

Total genomic DNA of all samples and controls was bisulfite treated converting unmethylated cytosines to uracil. By means of this treatment methylated cytosines are conserved. Bisulfite treatment was performed according to the applicant's optimised proprietary bisulfite treatment procedure. In order to avoid a potential process-bias, the samples were randomised into processing batches. In order to monitor the mPCR results ALF analysis was used.

### Hybridisation

All PCR products from each individual sample were then hybridised to glass slides carrying a pair of immobilised oligonucleotides for each CpG position under analysis. Each detection oligonucleotide was designed to hybridise to the bisulphite converted sequence around one CpG site which was either originally unmethylated (TG) or methylated (CG). See Table 2 for further details of all hybridisation oligonucleotides used (both informative and non-informative.) Hybridisation conditions were selected to allow the detection of the single nucleotide differences between the TG and CG variants.

Fluorescent signals from each hybridised oligonucleotide were detected using genepix scanner and software. Ratios for the two signals (from the CG oligonucleotide and the TG oligonucleotide used to analyse each CpG position) were calculated based on comparison of intensity of the fluorescent signals.

The samples were processed in batches of 80 samples randomised for sex, diagnosis, tissue, and bisulphite batch For each bisulfite treated DNA sample 2 hybridisation's were performed. This means that for each sample a total number of 4 chips were processed.

### Data Analysis

For the analysis of chip data, the applicant's proprietary software (‘Episcape’) was used. EpiScape contains a data warehouse that supports queries to sample, genome and laboratory management databases, respectively. It encompasses a variety of statistical tools for analysing and visualising methylation array data. In the following sections we summarise the most important data analysis techniques that were applied for analysing the data.

### From Raw Hybridisation Intensities to Methylation Ratios

The log methylation ratio (log(CG/TG)) at each CpG position is determined according to a standardised pre-processing pipeline that includes the following steps:
For each spot the median background pixel intensity is subtracted from the median foreground pixel intensity. This gives a good estimate of background corrected hybridisation intensities.
For both CG and TG detection oligonucleotides of each CpG position the background corrected median of the 4 redundant spot intensities is taken.
For each chip and each CG/TG oligo pair, the log(CG/TG) ratio is calculated.
For each sample the median of log(CG/TG) intensities over the redundant chip repetitions is taken.

This log ratio has the property that the hybridisation noise has approximately constant variance over the full range of possible methylation rates (see e.g. Huber W, Von Heydebreck A, Sultmann H, Poustka A, Vingron M. 2002. Variance stabilisation applied to Microarray data calibration and to the quantification of differential expression. Bioinformatics. 18 Suppl 1: S96-S104.)

### Comparison of Groups: Univariate Methods

Student paired sample t-test rank sum tests were used to compare groups (e.g. tumour vs. NAT) in terms of measurement values of single CpG sites. A significant test result (p<0.05) indicates a shift between the distributions of the respective methylation log ratios, i.e. log(CG/TG).

### Comparison of Groups: Multivariate Methods

As referred to herein a marker (sometimes also simply refereed to as gene or amplicon) is a genomic region of interest (also referred to herein using the abbreviation ROI). The ROI usually comprises several CpG positions. For testing the null hypothesis that a marker has no predictive power we use the likelihood ratio test for logistic regression models (see Venables, W. N. and Ripley, B. D. Modern Applied Statistics with S-PLUS, 3rd Ed. edition. New York: Springer, 2002). The logistic regression model for a single marker is a linear combination of methylation measurements from all CpG positions in the respective ROI. The fitted logistic regression model is compared to a constant probability model that is independent of methylation and represents the null hypothesis. The p-value of the marker is computed via the likelihood ratio test.

A significant p-value for a marker means that the methylation of this ROI has some systematic correlation to the question of interest as given by the sample classes. In general a significant p-value does not necessarily imply a good classification performance. However, because with logistic regression we use a linear predictor as the basis of our test statistic small p-values will be indicative of a good clinical performance.

### Multiple Test Corrections

Performing a large number of tests at the 5% level will lead to a large number of false positive test results. If there are no differences between groups, the probability of rejecting at least one hypothesis of equality is nearly 1, if about 200 tests are performed. Correction for multiplicity is therefore necessary to reliably conclude that a test result is really significant. A conservative, but simple method is the Bonferroni correction which multiplies all p-values by the number of tests performed, where corrected values >1 are censored to 1.0.

Bonferroni corrections are used for all analyses. The correction helps to avoid spurious findings, however, it is a very conservative method and false negative results (“missed markers”) are a frequent consequence. Therefore, results corrected by the less conservative False Discovery Rate (FDR) methods are also given.

### Class Prediction by Supervised Learning

In order to give a reliable estimate of how well the CpG ensemble of a selected marker can differentiate between different tissue classes it is possible to determine its prediction accuracy by classification. For that purpose it is necessary to calculate a methylation profile-based prediction function using a certain set of tissue samples with a specific class label. This step is called training and it exploits the prior knowledge represented by the data labels. The prediction accuracy of that function is then tested on a set of independent samples. A method of choice is the support vector machine (SVM) algorithm (see e.g. Cristiannini, N. and Shawe-Taylor, J. An introduction to support vector machines. Cambridge, UK: Cambridge University Press, 2000; Duda, R. O., Hart, P. E., and Stork, D. G. Pattern Classification. New York: John Wiley and Sons, 2001) to learn the prediction function.

### Results

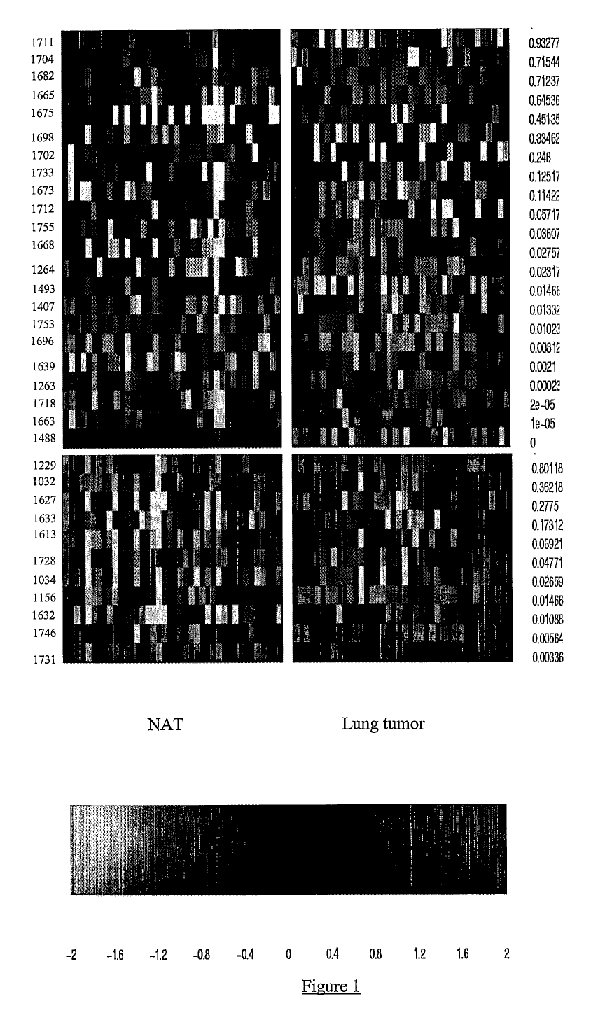

For each of the analyses results are provided in tables 4-11. Mean methylation signal is provided along with the variance thereof. FIGS. 1-8 are matrices showing a calibrated representation of the level of methylation at each CpG position within each sample.

Lung Cancer vs. Normal Lung Adjacent Tissue

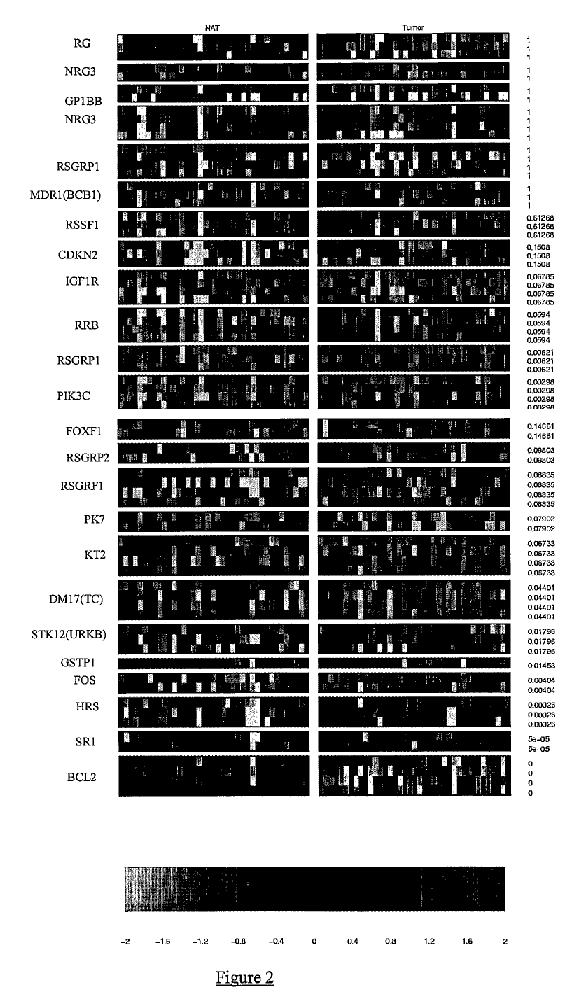

The lung cancer group consisted of both adenocarcinoma and squamous cell carcinoma. See table 4 for univariate results and table 5 for multivariate results. FIG. 1 shows the ranked matrices of the data obtained according to CpG methylation differences between the two classes of tissues using a univariate analysis. FIG. 2 shows a ranked matrices of the data obtained according to CpG methylation differences between the two classes of tissues using a multivariate analysis. The most significant CpG positions are at the bottom of the matrix with significance decreasing towards the top. Red indicates total methylation at a given CpG position, green represents no methylation at the particular position. Each row represents one specific CpG position of a gene and each column shows the methylation profile for the different CpGs for one sample.

Using FDR correction methylation differences that were considered significant were found either by univariate or multivariate analysis in a total of 36 genes. These were AKT2, BCL2, CDKN2A, ERBB4, FOS, GSTP1, HRAS, MDR1(ABCB1), RARB, ESR1, BCL2, PIK3CA, MAPK1, EREG, RASSF1, IGF1R, EGF, IGF1, STAT1, AREG, GP1BB, PTPRCAP(RPS6KB2), STK15(STK6), BTC, FOXF1, SHC1, STK12(AURKB), RASGRP2, ABCC4, RASGRP1, RASGRF1, MVP, ADAM17(TACE), ABCC12, PAK7 and NRG3.

Two genes identified in univariate analysis did not show significant differences in multivariate analysis while eight genes that were not identified as significantly different in univariate analysis showed up in the multivariate analysis.

Lung Adenocarcinoma vs. Lung Squamous Cell Carcinoma

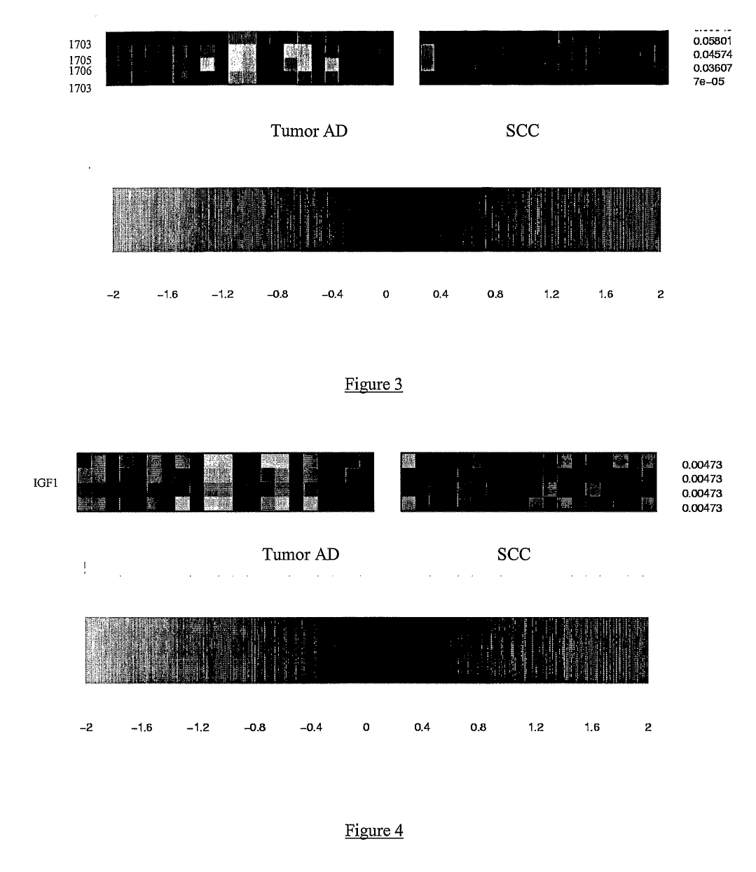

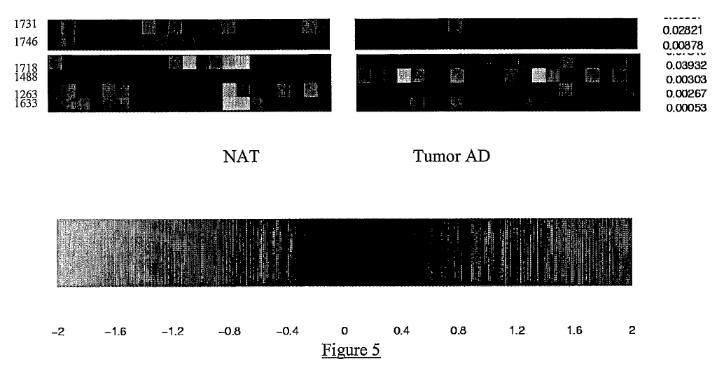

See Table 6 for univariate results and Table 7 for multivariate results. FIG. 3 shows a ranked matrices of the data obtained according to CpG methylation differences between the two classes of tissues using a univariate analysis. FIG. 4 shows a ranked matrices of the data obtained according to CpG methylation differences between the two classes of tissues using a multivariate analysis. Results from each of the two microarrays is shown as a separate matrix of each figure. The most significant CpG positions are at the bottom of the matrix with significance decreasing towards the top. Red indicates total methylation at a given CpG position, green represents no methylation at the particular position. Each row represents one specific CpG position of a gene and each column shows the methylation profile for the different CpGs for one sample. Only the gene IGFR1 was found to be significant in either of univariate or multivariate Bonferroni corrected methods.

Lung Adenocarcinoma vs. Normal Adjacent Tissue

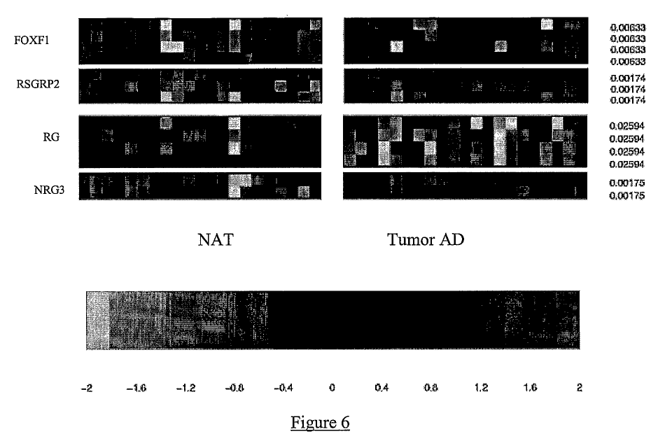

See Table 8 for univariate results and Table 9 for multivariate results. FIG. 5 shows a ranked matrices of the data obtained according to CpG methylation differences between the two classes of tissues using a univariate analysis. FIG. 6 shows a ranked matrices of the data obtained according to CpG methylation differences between the two classes of tissues using a multivariate analysis. Results from each of the two microarrays is shown as a separate matrix of each figure. The most significant CpG positions are at the bottom of the matrix with significance decreasing towards the top. Red indicates total methylation at a given CpG position, green represents no methylation at the particular position. Each row represents one specific CpG position of a gene and each column shows the methylation profile for the different CpGs for one sample. After Bonferroni correction the genes AREG, GP1BB, FOXF1, RASGRP2 and NRG3 were significant in differentiating between the two classes.

Lung Squamous Cell Carcinoma vs. Normal Lung Adjacent Tissue

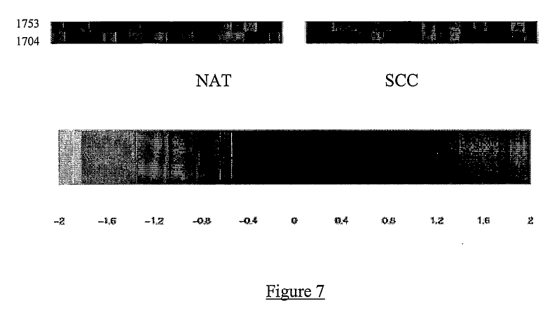

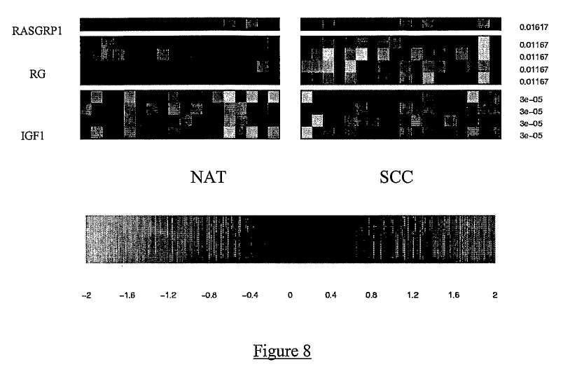

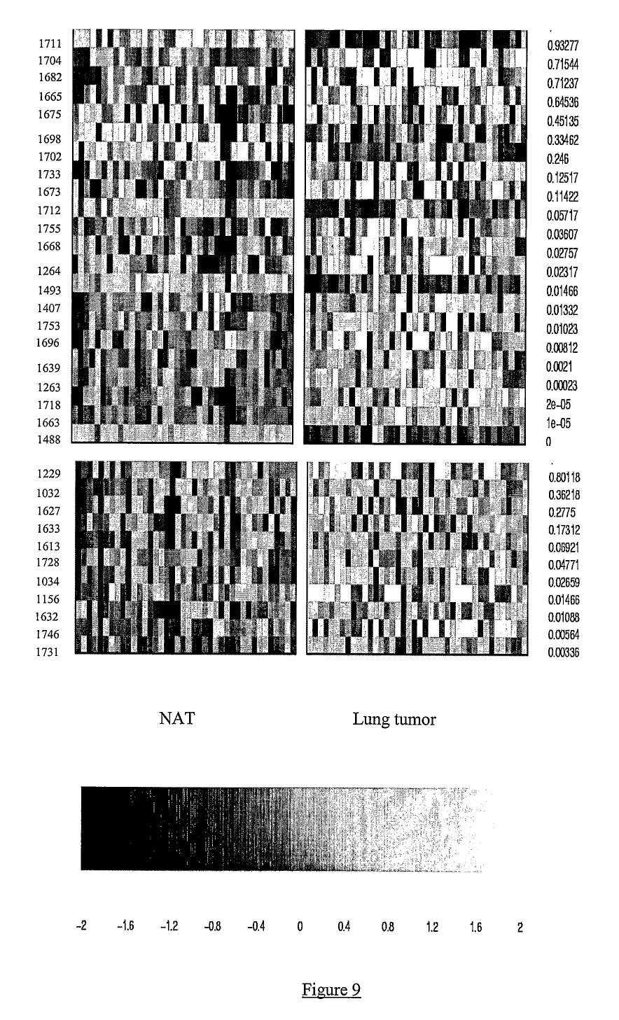

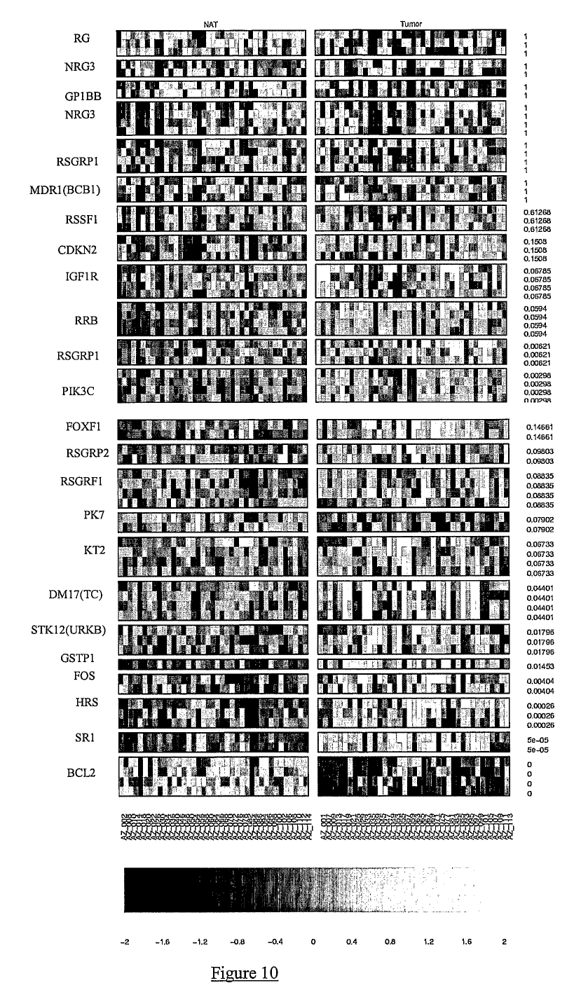

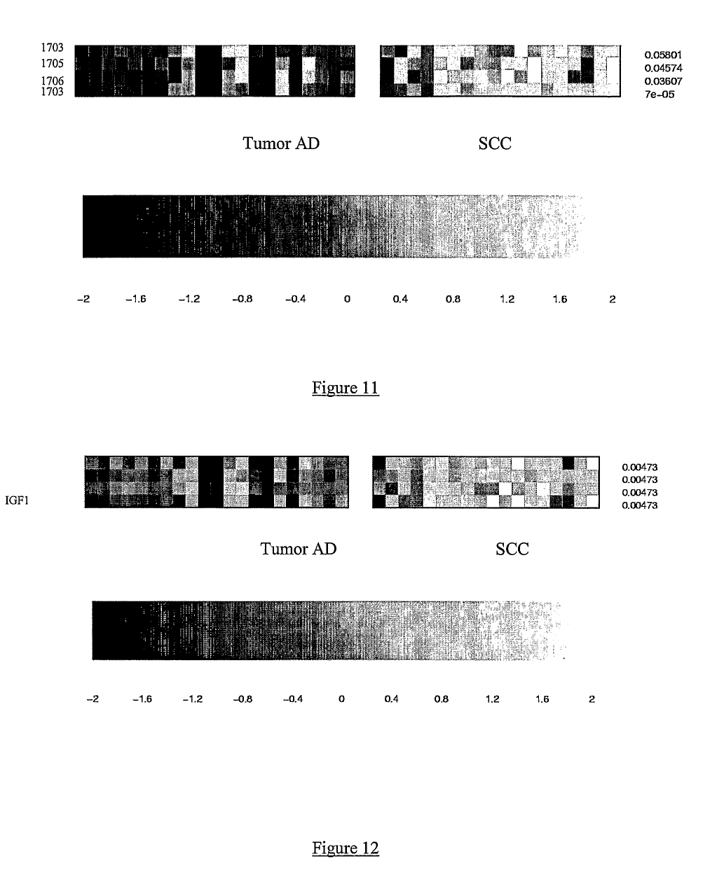

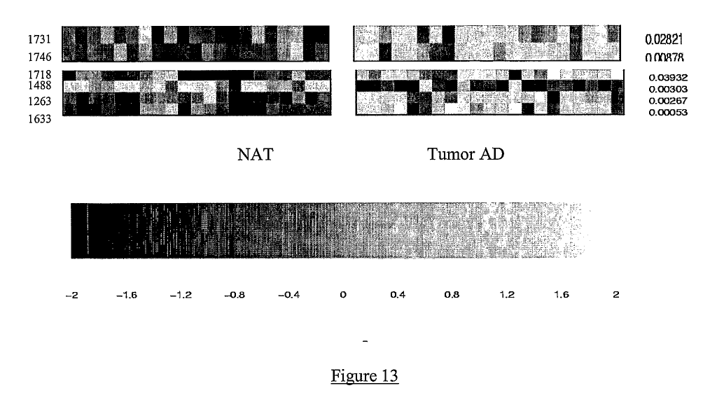

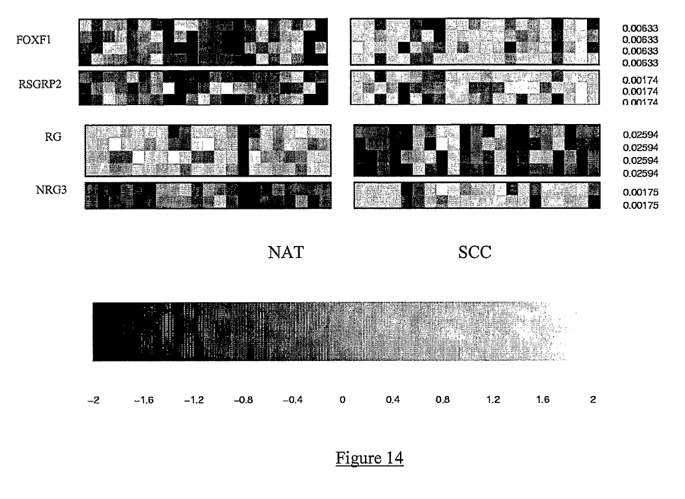

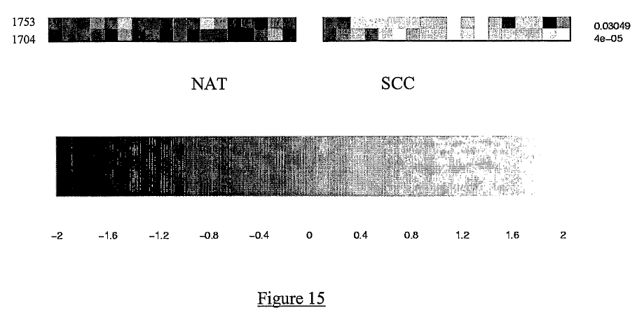

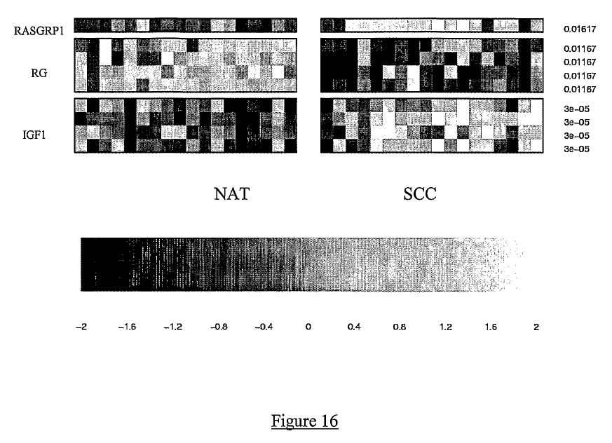

See Table 10 for univariate results and Table 11 for multivariate results. FIG. 7 shows a ranked matrices of the data obtained according to CpG methylation differences between the two classes of tissues using a univariate analysis. FIG. 8 shows a ranked matrices of the data obtained according to CpG methylation differences between the two classes of tissues using a multivariate analysis. Results from each of the two microarrays is shown as a separate matrix of each figure. The most significant CpG positions are at the bottom of the matrix with significance decreasing towards the top. Red indicates total methylation at a given CpG position, green represents no methylation at the particular position. Each row represents one specific CpG position of a gene and each column shows the methylation profile for the different CpGs for one sample. After Bonferroni correction the genes IGF1, AREG and RASGRP1 were significant in differentiating between the two classes.

1. A method for detecting, or for detecting and distinguishing between or among lung cell proliferative disorders in a subject, comprising determining the expression of at least one gene selected from the group consisting of AKT2, BCL2, CDKN2A, ERBB4, FOS, GSTP1, HRAS, MDR1(ABCB1), RARB, ESR1, BCL2 μl, PIK3CA, MAPK1, EREG, RASSF1, IGF1R, EGF, IGF1, STAT1, AREG, GP1BB, PTPRCAP(RPS6KB2), STK15(STK6), BTC, FOXF1, SHC1, STK12(AURKB), RASGRP2, ABCC4, RASGRP1, RASGRF1, MVP, ADAM17(TACE), ABCC12, PAK7 and NRG3 respectively and concluding therefrom upon the presence or absence of a lung cell proliferative disorder and/or distinguishing between or among lung cell proliferative disorders.

2. The method according to claim 1 wherein the presence or absence of lung cancer is determined.

3. A method according to claim 1 wherein lung squamous cell carcinoma is differentiated from lung adenocarcinoma and wherein the expression of the gene IGF1 is determined.

4. A method according to claim 1 wherein lung squamous cell carcinoma is detected and wherein the expression of at least one gene selected from the group consisting of IGF1, AREG and RASGRP1 is determined.

5. A method according to claim 1 wherein lung adenocarcinoma is detected and wherein the expression of at least one gene selected from the group consisting of AREG, GP1BB, FOXF1, RASGRP2 and NRG3 is determined.

6. A method according to any of claims 1 to 5 wherein said expression is determined by means of CpG methylation.

7. A method according to any of claims 1 to 5 wherein said expression is determined by means of mRNA or protein expression.

8. A method according to any of claims 1 to 6, comprising contacting genomic DNA isolated from a biological sample obtained from the subject, with at least one reagent, or series of reagents that distinguishes between methylated and non-methylated CpG dinucleotides within at least one target region of the genomic DNA, wherein the target region comprises, or hybridises under stringent conditions to a sequence of at least 16 contiguous nucleotides of at least of the genes wherein said contiguous nucleotides comprise at least one CpG dinucleotide sequence, and whereby detecting, or detecting and distinguishing between or among lung cell proliferative disorders is, at least in part, afforded.

9. A method according to claim 8 comprising
a. extracting or otherwise isolating genomic DNA from a biological sample obtained from the subject;
b. treating the genomic DNA of a), or a fragment thereof, with one or more reagents to convert cytosine bases that are unmethylated in the 5-position thereof to uracil or to another base that is detectably dissimilar to cytosine in terms of hybridization properties;
c. contacting the treated genomic DNA, or the treated fragment thereof, with an amplification enzyme and at least two primers comprising, in each case a contiguous sequence of at least 9 nucleotides that is complementary to, or hybridizes under moderately stringent or stringent conditions to a sequence selected from the group consisting of SEQ ID NO: 236 to SEQ ID NO: 347, and complements thereof, wherein the treated genomic DNA or the fragment thereof is either amplified to produce at least one amplificate, or is not amplified; and
d. determining, based on a presence or absence of, or on a property of said amplificate, the methylation state of at least one CpG dinucleotide of a sequence selected from the group consisting SEQ ID NO: 1 to SEQ ID NO: 56, or an average, or a value reflecting an average methylation state of a plurality of CpG dinucleotides of a sequence selected from the groups consisting of SEQ ID NO: 1 to SEQ ID NO: 56, whereby at least one of detecting, or detecting and distinguishing between lung cell proliferative disorders is, at least in part, afforded.

10. The method of claim 9, wherein treating the genomic DNA, or the fragment thereof in b), comprises use of a reagent selected from the group consisting of bisulfite, hydrogen sulfite, disulfite, and combinations thereof.

11. The method of claim 9, wherein contacting or amplifying in c) comprises use of at least one method selected from the group consisting of: use of a heat-resistant DNA polymerase as the amplification enzyme; use of a polymerase lacking 5′-3′ exonuclease activity; use of a polymerase chain reaction (PCR); generation of an amplificate nucleic acid molecule carrying a detectable labels; and combinations thereof.

12. The method of claim 9, wherein the biological sample obtained from the subject is selected from the group consisting of cell lines, histological slides, biopsies, paraffin-embedded tissue, bodily fluids, sputum, blood plasma, blood serum, whole blood, isolated blood cells, cells isolated from the blood and all possible combinations thereof.

13. The method of claim 9, further comprising in step d) the use of at least one nucleic acid molecule or peptide nucleic acid molecule comprising in each case a contiguous sequence at least 9 nucleotides in length that is complementary to, or hybridizes under moderately stringent or stringent conditions to a sequence selected from the group consisting of SEQ ID NO: 236 to SEQ ID NO: 347, and complements thereof, wherein said nucleic acid molecule or peptide nucleic acid molecule suppresses amplification of the nucleic acid to which it is hybridized.

14. A treated nucleic acid derived from genomic SEQ ID NO: 1 to SEQ ID NO: 56 wherein the treatment is suitable to convert at least one unmethylated cytosine base of the genomic DNA sequence to uracil or another base that is detectably dissimilar to cytosine in terms of hybridization.

15. A nucleic acid, comprising at least 16 contiguous nucleotides of a treated genomic DNA sequence selected from the group consisting of SEQ ID NO: 236 to SEQ ID NO: 347, and sequences complementary thereto, wherein the treatment is suitable to convert at least one unmethylated cytosine base of the genomic DNA sequence to uracil or another base that is detectably dissimilar to cytosine in terms of hybridization.

16. The nucleic acid of claims 14 and 15 wherein the contiguous base sequence comprises at least one CpG, TpG or CpA dinucleotide sequence.

17. The nucleic acid of claims 14 and 15 wherein the treatment comprises use of a reagent selected from the group consisting of bisulfite, hydrogen sulfite, disulfite, and combinations thereof.

18. A kit comprising a bisulfite reagent as well as oligonucleotides and/or PNA-oligomers having a length of at least 16 nucleotides which hybridizes to a pretreated nucleic acid sequence according to one of SEQ ID NO: 236 to SEQ ID NO: 347 and sequences complementary thereto, wherein the base sequence of said oligomers comprises at least one CpG, CpA or TpG dinucleotide.
